(19) United States
(12) Patent Application Publication
(10) Pub. No.: US 2022/0299513 A1
(43) Pub. Date: Sep. 22, 2022

(54) IRF2 AS A PROGNOSTIC BIOMARKER AND TARGET FOR AUGMENTING IMMUNOTHERAPY

(71) Applicant: University of Massachusetts, Boston, MA (US)

(72) Inventors: Kenneth L. Rock, Chestnut Hill, MA (US); Barry Alan Kriegsman, Holliston, MA (US)

(21) Appl. No.: 17/608,890

(22) PCT Filed: May 11, 2020
(86) PCT No.: PCT/US2020/032366

§ 371 Date: Nov. 4, 2021

Related U.S. Application Data
(60) Provisional application No. 62/846,244, filed on May 10, 2019.

(51) Int. Cl. G01N 33/574; A61K 31/167; A61K 31/706

(52) U.S. Cl. CPC G01N 33/57407; A61K 31/167; A61K 31/706; G01N 2800/52

(57) ABSTRACT

Methods of identifying and treating subjects with immune checkpoint inhibitors and interferon inducers.

### CLAIM OF PRIORITY

This application claims the benefit of U.S. Provisional Patent Application Ser. No. 62/846,244, filed on May 10, 2019. The entire contents of the foregoing are hereby incorporated by reference.

### FEDERALLY SPONSORED RESEARCH OR DEVELOPMENT

This invention was made with Government support under Grant No. AI114495 awarded by the National Institutes of Health. The Government has certain rights in the invention.

### TECHNICAL FIELD

Described herein are methods of identifying and treating subjects with immune checkpoint inhibitors and interferon inducers.

### BACKGROUND

The importance of adaptive immunity in preventing cancer was revealed through studies in which immunodeficient animals, such as those lacking IFNγ, perforin, or RAG-2, were found to have a marked increase in spontaneous and mutagen-induced tumors¹⁻³. In addition, tumors derived from such immunodeficient animals grew when transplanted into other immunodeficient hosts but were rejected when placed into immunocompetent hosts³⁻⁵, providing further evidence that the immune system recognized such tumors and could reject them. In contrast, many tumors arising in immunocompetent animals grew after being transplanted into immunocompetent hosts³⁻⁵, thereby showing that cancers that arise and successfully progress in the face of the immune system have undergone immunoediting to escape from immune control. This immunoediting process is thought to be why many cancers express low levels of MHC-I and upregulate certain inhibitory molecules⁶. The underlying molecular mechanisms responsible for these changes are poorly understood but have obvious potential impact on tumor progression and immunotherapy^{7,8}.

### SUMMARY

The invention is based, at least in part, on the discovery of an immune evasion mechanism that is commonly used by many human cancers—specifically, loss of interferon regulatory factor 2 (IRF2) expression. We have defined the underlying mechanisms for how IRF2 causes these effects. Measuring IRF2 levels in tumors could be used as a test to identify patients that will benefit or not from checkpoint blockade therapy. Restoring IRF2 expression in cancers could be a therapeutic approach for immunotherapy, either alone or in conjunction with checkpoint blockade. We have also discovered that reversing the effects of the loss of IRF2, e.g., by treatment of cells with either type I or type 2 interferons or other inducers of interferons (e.g. polylC), has a beneficial effect.

Thus, provided herein are methods for treating a subject who has cancer. The methods include providing a sample comprising cells from the cancer; detecting a level of a biomarker selected from the group consisting of interferon regulatory factor 2 (IRF2); transporter 2, ATP binding cassette subfamily B membertransporter 2, ATP binding cassette subfamily B member (TAP2); and endoplasmic reticulum aminopeptidase 1 (ERAP1) in the sample; comparing the level of the biomarker to a reference level; and (i) identifying a subject as having biomarker levels above the reference level, and treating the subject with a checkpoint inhibitor; (ii) identifying a subject as having biomarker levels below the reference level, and treating the subject with a checkpoint inhibitor and an interferon inducer and/or epigenetic modifier; or (iii) identifying a subject as having biomarker levels below the reference level, and treating the subject with a treatment that does not include a checkpoint inhibitor.

Also provided herein are methods for selecting a treatment for a subject who has cancer. The methods include providing a sample comprising cells from the cancer; detecting a level of a biomarker selected from the group consisting of interferon regulatory factor 2 (IRF2); transporter 2, ATP binding cassette subfamily B membertransporter 2, ATP binding cassette subfamily B member (TAP2); and endoplasmic reticulum aminopeptidase 1 (ERAP1) in the sample; comparing the level of the biomarker to a reference level; and (i) identifying a subject as having biomarker levels above the reference level, and selecting a treatment for the subject comprising administering a checkpoint inhibitor; (ii) identifying a subject as having biomarker levels below the reference level, and selecting a treatment for the subject comprising administering a checkpoint inhibitor and an interferon inducer and/or epigenetic modifier; or (iii) identifying a subject as having biomarker levels below the reference level, and selecting a treatment for the subject with a treatment that does not include a checkpoint inhibitor.

Additionally provided herein are methods for predicting response to treatment with a checkpoint inhibitor in a subject with cancer. The methods include providing a sample comprising cells from the cancer; detecting a level of a biomarker selected from the group consisting of interferon regulatory factor 2 (IRF2); transporter 2, ATP binding cassette subfamily B member transporter 2, ATP binding cassette subfamily B member (TAP2); and endoplasmic reticulum aminopeptidase 1 (ERAP1) in the sample; comparing the level of the biomarker to a reference level; and predicting that the subject will respond to the treatment if the level of the biomarker in the sample is above the reference level.

Further, provided herein are method for selecting treatment with a checkpoint inhibitor for a subject with cancer. The methods include providing a sample comprising cells from the cancer; detecting a level of a biomarker selected from the group consisting of interferon regulatory factor 2 (IRF2); transporter 2, ATP binding cassette subfamily B membertransporter 2, ATP binding cassette subfamily B member (TAP2); and endoplasmic reticulum aminopeptidase 1 (ERAP1)in the sample; comparing the level of the biomarker to a reference level; and selecting the treatment for the subject if the level of the biomarker in the sample is above the reference level.

In addition, provided herein are methods for treating a subject with cancer with a checkpoint inhibitor. The methods include providing a sample comprising cells from the cancer; detecting a level of a biomarker selected from the group consisting of interferon regulatory factor 2 (IRF2); transporter 2, ATP binding cassette subfamily B membertransporter 2, ATP binding cassette subfamily B member (TAP2); and endoplasmic reticulum aminopeptidase 1 (ERAP1) in the sample; comparing the level of the biomarker to a reference level; and treating the subject with the checkpoint inhibitor if the level of the biomarker in the sample is above the reference level.

In some embodiments, the methods described herein include detecting a level of programmed cell death 1 ligand 1 (PD-L1) in the sample; comparing the level of PD-L1 to a reference level; and selecting and optionally treating the subject with the checkpoint inhibitor if the level of the biomarker in the sample is above the reference level, and the level of PD-L1 is above the reference level, or selecting and optionally treating the subject with the checkpoint inhibitor and an interferon inducer and/or epigenetic modifier if the level of the biomarker in the sample is below the reference level, and the level of PD-L1 is above the reference level, or selecting and optionally treating the subject with a treatment that does not include a checkpoint inhibitor if the level of the biomarker in the sample is below the reference level, and the level of PD-L1 is below the reference level.

In some embodiments of the methods described herein, detecting a level of the biomarker in the sample comprises measuring mRNA or protein levels in the sample.

In some embodiments of the methods described herein, measuring mRNA comprises using quantitative PCR.

In some embodiments of the methods described herein, the subject has a carcinoma or adenocarcinoma. In some embodiments, the carcinoma or adenocarcinoma is breast carcinoma, cholangiocarcinoma, colon adenocarcinoma, liver hepatocellular carcinoma, lung adenocarcinoma, squamous cell carcinoma (non-small cell lung cancer), prostate adenocarcinoma, rectum adenocarcinoma, stomach adenocarcinomas, or uterine carcinoma.

In some embodiments of the methods described herein, the checkpoint inhibitor is an antibody that targets programmed cell death protein 1 (PD-1), PD-Ligand 1(PD-L1), PDL2, or cytotoxic T-lymphocyte-associated protein 4 (CTLA-4).

In some embodiments of the methods described herein, the interferon inducer is type I or type 2 interferon. In some embodiments, the interferon is interferon alpha-2b, PEGylated interferon alpha-2b, PEGylated interferon-alpha-2a, Human leukocyte Interferon-alpha (HuIFN-alpha-Le), Interferon beta 1a, Interferon beta 1b, or Interferon gamma (e.g., IFN-gamma 1b).

In some embodiments, the interferon inducer is poly(I:C), Poly(A:U), ampligen [poly(I)-poly(Cl2U)], polyICLC), or Imiquimod.

In some embodiments of the methods described herein, the epigenetic modifier is a DNA methyltransferase (DNMT) inhibitor or a Histone deacetylase (HDAC) inhibitor. In some embodiments, the methods include administering both a DNMT inhibitor and an HDAC inhibitor.

In some embodiments, the HDAC inhibitor is Suberoylanilide hydroxamic acid (SAHA/Vorinostat/Zolinza), Trichostatin A (TSA), belinostat (PXD101), depsipeptide (FK228/romidepsin/ISTODAX), Entinostat (SNDX-275), mocetinostat (MGCD0103), valproic acid, Sodium phenylbutyrate, LAQ824, panobinostat (LBH589), entinostat (MS275), CI-994 (N-acetyldinaline/tacedinaline), EVP-0334, SRT501, CUDC-101, JNJ-26481585, PCI24781, or Givinostat (ITF2357).

In some embodiments, the DNMT inhibitor is 5′-azacytidine (Aza), Decitabine, Cladribine, Fludarabine, Clofarabine, Procainamide, Procaine, Zebularine (1-(β-D-ribofuranosyl)-1,2-dihydropyrimidin-2-one), (−)-epigallocatechin-3-gallate, MG98, hydralazine, RG108, or chlorogenic acid.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-I. IRF2 positively regulates MHC-I presentation under basal conditions. (A) Representative histograms of surface MHC I levels by W6/32 staining in HeLa H1 lines stably transduced with the LentiCRISPRv2 constructs; “IRF2 KO” (IRF2 sgRNA) or “WT” control (no sgRNA). Background is with secondary antibody staining only; (B) Normalized MFI of surface MHC I levels on HeLa H1 (left) or HEK293T (right) stable knockout lines; (C) normalized MFI of surface MHC I levels on HeLa H1 (left) or HEK293T (right) cells after 72 hrs silencing with 10 nM indicated siRNA; (D) Normalized MFI of surface MHC I levels by AF6 staining on 3T3-K^{b} stable knockout lines (left), surface MHC I levels on DC3.2 stable knockout lines (middle), or surface MHC II levels on DC3.2 stable knockout lines (right); (E) Representative western blot in DC3.2 lines “IRF2 KO” (IRF2 sgRNA) or “WT” control (no sgRNA) for protein expression of IRF2 or β-actin as control; (F) Representative cross-presentation experiment of OVA-beads by DC3.2 lines to RF33.70-Luc CD8⁺ T cell hybridoma; (G) Representative MHC-II presentation experiment of OVA-beads by DC3.2 lines to MF2.2D9-Luc CD4⁺ T cell hybridoma; (H) Normalized MFI of surface MHC I levels on DC3.2 lines after transducing with pCDH expressing empty vector (EV) or wild-type IRF2 containing six synonymous mutations within the IRF2 sgRNA target site (IRF2); (I) Cross-presentation after transducing the DC3.2 lines with pCDH expressing empty vector (EV), wild-type IRF2 containing six synonymous mutations within the IRF2 sgRNA target site (IRF2 WT), or mutant IRF2 (IRF2 K78R) which also contains the same six synonymous mutations as IRF2 WT. (B, C, D, H, I) Bars represent mean+SEM (N≥3). Statistical analysis by two-tailed ratio paired t-tests; (F, G) Points represent mean±SD of technical duplicates. *p<0.05, **p<0.01, ***p<0.001, ns=not significant

FIGS. 2A-C. IRF2-mediated transcriptional regulation of MHC-I pathway under basal conditions. (A) Heatmap of genes (52) differentially expressed by >2-fold between the DC3.2 no sgRNA (“WT”) and IRF2 sgRNA (“KO”) lines. Columns represent independent duplicate RNA-seq runs in these two lines. Top left are high, top right are low; bottom left are low, bottom right are high. For clarity, only three of the downregulated genes in the KO line (Psme1, Tap2, and Erap1) are shown; all of the genes are listed in Table 1; (B) mRNA expression levels by qPCR in DC3.2 IRF2 KO relative to those in DC3.2 WT; normalized to the mRNA expression of mouse β-actin in each sample (2\hat{ }^{-ΔΔCt}). Values>1 indicate higher expression in DC3.2 IRF2 KO and values<1 indicate lower expression in the DC3.2 IRF2 KO. Bars represent mean+SEM mRNA expression (N≥3). Statistical analysis by two-tailed unpaired t-tests by comparing the expression of a given gene to that of H2-Ab1 (control); (C) ChIP-qPCR in DC3.2 WT for TAP2 (left) or ERAP1 (right) DNA with rabbit anti-IRF2 IgG or normal rabbit IgG (control). Bars represent mean +SEM fold enrichment (2\hat{ }^{ΔCt}) over the normal rabbit IgG control IP (N=2). Statistical analysis by two-tailed ratio paired t-tests. *p<0.05, **p<0.01, ***p<0.001, ns=not significant.

TABLE 1

| Cluster 1 (top bar) | Cluster 2 (bottom bar) |
|---|---|
| Isg20 | Socs1 |
| Arg1 | Ifit1 |
| Acadl | H2-T22 |
| Ddx58 | Hck |
| Thbs1 | Igtp |
| C1qa | Flt1 |
| Psme1 | Mx2 |
| Fabp7 | Tnfsf13b |
| Nmi | H2-T9 |
| Arg2 | Ly6a |
| Epsti1 | Scimp |
| Mov10 | Tmem140 |
| Il15ra | Cmpk2 |
| Casp7 | Gbp5 |
| Gsdmd | Rsad2 |
| Parp14 | Gbp2 |
| Ifi27 | Gbp6 |
| Tap2 | Cd40 |
| Erap1 | Gbp11 |
| | Trafd1 |
| | Psmb8 |
| | Irg1 |
| | Cst7 |
| | Tspan13 |
| | Gbp3 |
| | Ifi47 |
| | Gbp8 |
| | Irgm2 |
| | Fgl2 |
| | Tgtp2 |
| | Gbp9 |
| | Gbp4 |
| | Gbp7 |

FIGS. 3A-E. IRF2 affects antigen transport and processing. (A-C) H-2K^{b} presentation of SIINFEKL derived from (A) the TAP-dependent, ERAP1-dependent antigens CD16-OVA (left), N25-S8L (middle), or N5-S8L (right); (B) the TAP-independent, ERAP1-dependent antigen ss-N5-S8L; and (C) the TAP-independent, ERAP1-independent antigen ss-S8L on 3T3-K^{b} IRF2-knockout lines. 25-D1.16 staining analyzed on transfected (GFP+) cells; (D) Normalized MFI of surface MHC I levels on 3T3-K^{b} (left) or DC3.2 (right) knockout lines; (E) Normalized MFI of surface MHC I levels on DC3.2 lines 48 hrs after transduction with pCDH expression vectors containing empty vector (EV), IRF2, TAP2, ERAP1, or dual transduction of TAP2 and ERAP1. (A-E) Bars represent the mean +SEM of the normalized MFI from independent experiments (N≥3). Statistical analysis by two-tailed ratio paired t-tests. *p<0.05, ***p<0.001, ns=not significant

FIGS. 4A-D. IRF2 represses PD-L1 expression under basal conditions. (A) H2-Ab1 and PD-L1 mRNA expression levels by qPCR in DC3.2 IRF2 KO relative to those in DC3.2 WT; normalized to the mRNA expression of mouse β-actin in each sample (2\hat{ }^{-ΔΔCt}). Values >1 indicate higher expression in DC3.2 IRF2-KO. Bars represent mean+SEM mRNA expression (N=3). Statistical analysis by two-tailed unpaired t-test by comparing the expression of PD-L1 to that of H2-Ab1 (control); (B) ChIP-qPCR of DC3.2 WT for PD-L1 DNA with rabbit anti-IRF2 IgG or normal rabbit IgG (control); bars represent mean +SEM fold enrichment (2\hat{ }^{ΔCt}) over the normal rabbit IgG control IP (N=2). Statistical analysis by two-tailed ratio paired t-test; (C) Representative histograms of surface PD-L1 levels by 10F.9G2 staining in DC3.2 lines stably transduced with the LentiCRISPRv2 constructs; “IRF2 KO” (IRF2 sgRNA) or “WT” control (no sgRNA). Background=isotype control staining; (D) Normalized MFI of surface PD-L1 levels on DC3.2 lines; bars represent mean+SEM (N=6). Statistical analysis by two-tailed ratio paired t-test. *p<0.05, **p<0.01

FIGS. 5A-E. IRF2-IRF1 balance. (A) Normalized MFI of surface MHC I and PD-L1 levels on the indicated single or double (IRF1+IRF2, double knock out (DKO)) DC3.2 knockout lines after overnight incubation with either media alone or 2 ng/mL IFNγ; bars represent mean+SEM (N≥3); (B) Western blot for IRF1 and IRF2 (and β-actin as loading control) in the DC3.2 knockout lines in the absence or presence of 2 ng/mL IFNγ for the durations indicated; (C) ChIP-qPCR of DC3.2 WT cells stimulated with ±2 ng/mL IFNγ for 2 hrs for TAP2 (top), ERAP1 (middle), or PD-L1 (bottom) DNA with rabbit anti-IRF2 IgG, rabbit anti-IRF1 IgG, or normal rabbit IgG (control). Bars represent mean +SEM fold enrichment (2\hat{ }^{-ΔCt}) over the normal rabbit IgG control IP (N=2); (d, e) Heatmap of genes differentially expressed between the DC knockout lines (D) at baseline or (E) after stimulation with ±2 ng/mL IFNγ for 2 hrs. For clarity, only a few genes are shown. Statistical analysis by two-tailed ratio paired t-tests. *p<0.05, **p<0.01, ***p<0.001, ****p<0.0001, ns=not significant.

FIGS. 6A-J. IRF2 and cancer. (A) Differential IRF2 expression in tumor and normal tissue from patients with the indicated cancer types (TCGA abbreviations), as queried from TIMER²⁹; (B) IRF2 mRNA expression in patient NSCLC specimens scored as PD-L1 low (1-50%) or high (>50%) by immunohistochemistry. IRF2 mRNA expression was normalized to GAPDH mRNA expression in each lung specimen and then IRF2 expression across specimens was compared by calculating fold changes over the lowest IRF2-expressing specimen, which was set equal to 1. Statistical analysis by Mann-Whitney U test, **p<0.01; (C) TAP2 (top) and ERAP1 (bottom) mRNA correlations with IRF2 mRNA in NSCLC specimens. Linear regression models shown with R² for goodness of fit. (D-E) Geometric MFI of surface MHC I (left) and PD-L1 (right) on (D) unstimulated or (E) overnight IFNγ-stimulated A549 lines knocked out for IRF2 (“IRF2 KO”) or control (“WT”) or overexpressing IRF2 or empty vector (EV) as control. Bars represent mean +SEM (N=3). Statistical analysis by two-tailed ratio paired t-tests. *p<0.05, **p<0.01, ***p<0.001; (F) Geometric MFI of surface MHC-I and PD-L1 on unstimulated or IFNγ-stimulated human breast carcinoma (BT20 and MCF7) lines knocked out for IRF2 (“IRF2 KO”) or control (“WT”). Representative experiment shown (N=3), bars represent mean +SD of technical replicate staining; (G) Pre-activated OT-I effectors were cultured with pairs of wild-type (“WT RMA”) or IRF2 KO (“IRF2 KO RMA”) cells that were S8L-pulsed or not and labeled with different amounts of the dye CF SE. After 4 hours, specific killing was quantified by flow cytometry. (Left) % specific killing dose-titration curve for one representative experiment using an effector to target ratio of 1:2 and [S8L] indicated, points show mean±SD of technical triplicates; (Right) % specific killing of IRF2 KO RMA relative to that of WT RMA at 10 pM S8L; bars show mean+SEM (N=4); (H) Geometric MFI of surface MHC-I on prostate cancer cell lines Myc-cap (murine, left) and PC3 (human, right) cells either untreated (BKG) or transduced with vectors expressing Cas9 and a sgRNA targeting IRF2 or no sgRNA as control. The IRF2-knockout Myc-cap and PC3 cancers had significantly lower surface MHC-I levels than their wild-type counterparts; (I) Geometric MFI of surface MHC-I on human prostate cancer cell lines Myc-cap (left) and PC3 (right) were transduced with empty expression vector (EV) or the same vector containing IRF2. The IRF2-transduced Myc-cap and PC3 cancers had significantly higher surface MHC-I levels than ones transduced with EV; (J) Geometric MFI of surface MHC-I on SKMEL2 human melanoma cells transduced with empty expression vector (EV) or the same vector containing IRF2. The IRF2-transduced SKMEL2 cells had significantly higher surface MHC-I levels than ones transduced with EV. Statistical analysis by two-tailed ratio paired t-test, *p<0.05.

FIG. 7. TIDE analysis for IRF2 gene disruption in DC3.2 IRF2 KO cells (DC3.2 WT used as control).

FIG. 8. mRNA expression levels by qPCR in DC3.2 IRF2 KO cells 24hrs after transducing pCDH wild-type IRF2 (IRF2 WT) or mutant IRF2 (IRF2 K78R) relative to overexpressing pCDH empty vector; normalized to the mRNA expression of mouse β-actin in each sample (2\hat{ }-ΔΔCt). Values>1 indicate higher expression than overexpressing empty vector and values<1 indicate lower expression than overexpressing empty vector. Bars represent mean+SD of mRNA expression of duplicate technical replicates (N=1).

FIG. 9. PD-L1 mRNA expression in the DC3.2 no sgRNA (“WT”) and DC3.2 IRF2 sgRNA (“IRF2 KO”) lines. Bars represent TPM from 3 independent RNA-seq replicates.

FIGS. 10A-B. (A) Normalized MFI of surface MHC I and PD-L1 levels on DC3.2 stable knockout lines after overnight incubation with 5,000 U/mL IFNα; bars represent mean+SEM (N=3). Statistical analysis by two-tailed ratio paired t-tests. (B) Western blots of IRF1 and IRF2 (and β-actin as loading control) in DC3.2 KO lines in the absence or presence of 5,000 U/mL IFNα for the durations indicated.

FIGS. 11A-D. (A) RNA-seq of DC3.2 no sgRNA (WT), IRF2 sgRNA (IRF2 KO), IRF1 sgRNA (IRF1 KO), or IRF1 +IRF2 sgRNAs (DKO) after stimulation for 2hrs with 5,000U/mL IFNα or media alone. (B-D) Second RNA-seq replicate of FIG. 5D, 5E, and FIG. 11A, respectively.

FIGS. 12A-D. Other cell lines where the effects of IRF2 knockout or overexpression on surface MHC I and/or PD-L1 levels were tested. (A-D) Geometric MFI of (A) surface PD-L1 on IFNγ-stimulated BT20 and MCF7 knockout lines; (B) surface MHC-I (left) or surface PD-L1 (right) on unstimulated RMA lymphoma knockout lines; (C) surface MHC-I on (left) unstimulated BT20 and MCF7 lines or (right) unstimulated D53m and H50m mouse sarcoma lines overexpressing IRF2 (pCDH IRF2) or empty vector control (pCDH EV); and (D) surface PD-L1 on IFNγ-stimulated D53m and H50m overexpression lines. Representative experiments shown, error bars represent staining of technical replicates.

FIGS. 13A-B. (A) IRF2 & TAP2 mRNA levels, and MHC I surface protein levels in F221 murine sarcoma cells treated with Vorinostat (1 uM or 5 uM), Decitabine (5 uM), or control diluent (DMSO) with no drug; (B) surface expression of MHC I on F221 MCA-sarcoma cells treated with Vorinostat (5 uM) and/or Decitabine (5 uM).

### DETAILED DESCRIPTION

The major histocompatibility complex class I (MHC-I) presentation pathway is critical for immune recognition and elimination of tumors by CD8⁺ T cells. In this process, a fraction of peptides that are generated by proteasomal degradation of cellular proteins are transported by the TAP transporter into the endoplasmic reticulum (ER), wherein they can be further trimmed by the aminopeptidase, ERAP1⁹⁻¹¹. Subsequently, peptides of the correct length and sequence bind to MHC-I molecules and these complexes are then transported to the cell surface for display to CD8⁺ T cells. This allows activated CD8⁺ T cells to identify and kill cells that are presenting tumor-specific peptides (e.g., from mutant proteins) on their MHC-I¹².

As shown herein, IRF2 both positively regulates the MHC class I pathway and negatively regulates PD-L1 expression have implications for cancer progression and immunotherapy. Cancers need to evade the immune system and, if they are immunogenic, require editing to escape and progress⁶. One of the ways that murine tumors can evade immune elimination is by downregulating the MHC-I pathway³. Similar immunoediting of this pathway occurs in humans as it has been found that tumors, which were predominantly MHC-I positive at early stages, subsequently become homogeneously MHC-I deficient^{33,34} and progressing cancers are frequently MHC-I deficient^{35,36}. Another way tumors can evade immune elimination is by expressing checkpoint inhibitors. Indeed, many human cancers upregulate PD-L1 and those that do tend to be more aggressive and have fewer T cells present in the tumor³⁷. Blocking PD-L1 or its receptor (PD-1) can lead to tumor rejection in both mice and humans, proving that this is an important immune evasion mechanism^{38,39}. The molecular mechanisms that allow cancers to exploit these immune evasion strategies are incompletely understood but are important to elucidate for understanding pathogenesis, how they affect prognosis and immunotherapy, and how they might be reversed to improve therapy.

These studies have uncovered an immune evasion mechanism that is relatively frequent. Many cancers of diverse origins (NSCLC, breast, colorectal, liver, stomach, prostate, uterine) downregulate IRF2 expression, which is a molecule that is not essential for viability in fully differentiated cells. As a result of losing IRF2, tumor cells decrease their surface MHC-I expression and increase their surface PD-L1 expression. Thus, loss of a single gene can generate a “double whammy” for the immune system as it enables tumor cells lacking IRF2 to become both harder to identify (loss of MHC-I antigen presentation) and better able to suppress T cell-mediated elimination (increased checkpoint inhibition). As shown in in vitro cytotoxicity assay, the loss of IRF2 renders such tumor cells more difficult for CD8⁺ T cells to kill.

Analysis of IRF2-knockout cells revealed that PD-L1 was highly upregulated under basal conditions. It is well-established that IRF1 can promote IFNγ-inducible activation of PD-L1^{27,28}. Recently, Dorand et al. found that hyperphosphorylation of IRF2BP2 (an IRF2-binding protein) could lead to decreased PD-L1 expression after IFNγ stimulation⁴⁰. Additionally, Wu et al. recently discovered that a loss of IRF2BP2 can lead to enhanced IRF2 binding to the PD-L1 promoter and that IFNγ stimulation releases IRF2 from the PD-L1 promoter⁴¹. However, the role of IRF2 on PD-L1 expression had not been directly examined; based on the data, IRF2BP could be mediating its affects independently of IRF2. Our ChIP-qPCR data shows that IFNγ stimulation leads to both increased IRF1 binding and decreased IRF2 binding to the PD-L1 promoter. In addition, we show here for the first time that, under basal conditions, a loss of IRF2 significantly increases PD-L1 mRNA and surface expression and IRF2 overexpression produces the opposite effects. Collectively, these results establish the repressive role of IRF2 on PD-L1 expression in the absence and presence of IFNγ and reveal that unstimulated cells can upregulate PD-L1 expression if upstream regulatory factors, such as IRF2, are defective/absent.

The transcriptional control of many MHC class I pathway components, particularly under basal conditions, has not been well defined⁴². Herein, IRF2 was identified as a novel positive regulator of MHC-I antigen presentation and show that, under basal conditions, it was important for both classical MHC-I presentation and cross-presentation. IRF2 binds the promoters of TAP2 and ERAP 1 and transcriptionally activates their expression. In the absence of IRF2, cells expressed less TAP2 and ERAP1 and, due to the consequent defects in antigen transport and processing, present fewer peptide-MHC-I complexes at the cell surface. Additionally, IRF2 regulates the expression of other genes, such as immunoproteasome subunits, which likely also influence the MHC-I presentation in IRF2-deficient cells⁴³.

In the DC line where most of the studies were focused and in other cell lines described in the literature, IRF2 is constitutively expressed, relatively stable with a half-life of 8 hours, and minimally affected by IFN²³⁻²⁵. However, in these same sets of cells, IRF1 is minimally expressed under basal conditions but strongly induced in the presence of IFN and more short-lived than IRF2, with a half-life of only 0.5 hours²³. Due to these differences between IRF2 and IRF1, even though they both recognize and bind to the same ISRE²⁰⁻²², it was hypothesized that the relative contributions of IRF2 and IRF1 to certain signaling pathways varies depending on the inflammatory state of the cell. Although IRF1 and IRF2 were originally characterized as an activator and repressor of IFN-α/β expression, respectively²², several reports since then have highlighted that IRF1 and IRF2 do not always act as such^{24,44,45.} Yet, to the best of the present inventors' knowledge, there have been no global differential expression analyses in IRF1- and/or IRF2-lacking cells of the same cell type to better understand the synergistic vs. antagonistic roles these transcription factors perform. Here, RNA-seq was conducted on IRF1- and/or IRF2-knockout DCs in the absence or presence of IFN to help fill this void. Globally, the expression profile of the double knockout DCs differed from that of either IRF single knockout and that the relative contributions of IRF1 vs. IRF2, in terms of their ability to positively or negatively regulate certain genes, varied depending on the inflammatory state of the cell. Transcriptional control of genes can be highly cell-type dependent and therefore, it is possible that in other cells, the contribution of IRF2 on surface MHC-I and PD-L1 expression may vary. However, concordant observations in multiple mouse and human cell lines and primary tumors suggested that the number of cell types in which IRF2 regulates these genes in this manner is quite large.

Immune evasion due to tumor PD-L1 upregulation can be reversed by blocking the PD-L1/PD-1 interaction, which is the basis of targeted checkpoint blockade immunotherapy. However, any natural or invigorated (from checkpoint blockade) CD8⁺ T cell response to kill tumors that have downregulated their MHC-I expression will continue to be impaired. In this context, it is of considerable interest and potential importance that the downregulation of the MHC-I pathway from the loss of IRF2 is reversible. When IRF2-deficient cells were treated with IFN, MHC-I levels were restored, likely because of induction of IRF1 and possibly some other transcriptional activators. These findings suggest that interferons (which are FDA-approved for other indications) or interferon-inducing agents could be used to restore MHC-I antigen presentation in IRF2-low tumors. This would be predicted to enhance the effects of immunotherapies, such as checkpoint blockade, that are ultimately dependent on T cell receptor recognition of tumor MHC-I presentation. Currently, checkpoint therapy is effective in only some patients and, based on the mechanisms described herein, reversing the IRF2 defects may be used to increase the number of patients that can benefit. In addition, because checkpoint blockade is an extremely expensive therapy and one that can have serious side effects, there is a need for good biomarkers to identify those patients that would be more likely to benefit from this type of therapy.

### Subject Identification and Treatment Selection

Expression of IRF2 and its downstream target genes (e.g., TAP2 and ERAP1) can be used as biomarkers to help identify checkpoint blockade-responsive patients, e.g., to select patients who are predicted to respond to checkpoint inhibitors, and to identify subjects who would benefit from a combination treatment with an agent that increases levels of interferons and a checkpoint inhibitor. A treatment can then be selected for the subject based on the outcome of the prediction.

The methods rely on detection of IRF2 polypeptides or nucleic acids, including IRF2 proteins and mRNA. Exemplary sequences of IRF2 are provided in GenBank at Acc. Nos. NM_002199.4 (mRNA) and NP_002190.2 (protein). Alternatively or in addition, downstream target genes (e.g., TAP2 and ERAP1) can be used. Exemplary sequences of TAP2 are provided in GenBank at NM_000544.3 (mRNA) and NP_000535.3 (protein) for isoform 1; NM_018833.2 (mRNA) and NP_061313.2 (protein) for isoform 2; and NM_001290043.1 (mRNA) and NP_001276972.1 (protein) for isoform 3. Exemplary sequences of ERAP1 are provided in GenBank at NM_016442.4 (mRNA) and NP_057526.3 (protein) for isoform a precursor, variant 1; NM_001198541.2 (mRNA) and NP_001185470.1 (protein) for isoform b precursor, variant 3; NM_001040458.3 (mRNA) and NP_001035548.1 (protein) for isoform b precursor, variant 2; and NM_001349244.1 (mRNA) and NP_001336173.1 (protein) for isoform a precursor, variant 4. Variants 1 and 4 encode the same isoform (a), and variants 2 and 3 encode isoform (b). Exemplary sequences of PD-L1 (also known as CD274) are provided in GenBank at NM_014143.4 (mRNA) and NP_054862.1 (protein) for programmed cell death 1 ligand 1 isoform a precursor; NM_001267706.1 (mRNA) and NP_001254635.1 (protein) for programmed cell death 1 ligand 1 isoform b precursor; and NM_001314029.1 (mRNA) and NP_001300958.1 (protein) programmed cell death 1 ligand 1 isoform c precursor.

The methods include obtaining a sample from a subject, and evaluating the presence and/or level of IRF2 in the sample, and comparing the presence and/or level with one or more references, e.g., a control reference that represents a level of IRF2 in a subject who is expected to respond to checkpoint inhibitors without additional intervention as described herein, and/or a reference that represents a level of IRF2 associated with a subject who would not benefit from checkpoint inhibitors, or who may benefit from treatment with a checkpoint inhibitor and an agent that increases levels of interferons. In any of the present methods, alternatively or in addition to IRF2, downstream target genes (e.g., TAP2 and ERAP1) can be used.

The methods can also include measuring expression of PD-L1, e.g., evaluating the presence and/or level of PD-L1 in the sample, and comparing the presence and/or level with one or more references, e.g., a control reference that represents a level of PD-L1 in a subject who is expected to respond to checkpoint inhibitors without additional intervention as described herein, and/or a reference that represents a level of PD-L1 associated with a subject who would not benefit from checkpoint inhibitors, or who may benefit from treatment with a checkpoint inhibitor and an agent that increases levels of interferons. This in some embodiments, treatment selection is be based on whether IRF2 levels and PD-L1 levels are above or below a reference. If IRF2 and PD-L1 are above the reference ranges then PD-L1 checkpoint blockade would be chosen. If IRF2 is above the reference range and PDL-1 is below, then a non-PD-L1 checkpoint blockade would be chosen. If IRF2 is below the reference range and PD-L1 is above, then treatment with PD-L1 and an IRF2 inducer would be used. An exemplary decision grid is as follows:

| | Low PD-L1 | High-PD-L1 |
|---|---|---|
| Low IRF2 | Non-immunotherapy | PD-L1 Immunotherapy plus IFN |
| High IRF2 | Non-immunotherapy | PD-L1 Immunotherapy w/o IFN |

In any of the present methods, alternatively or in addition to IRF2, downstream target genes (e.g., TAP2 and ERAP1) can be used.

As used herein the term “sample”, when referring to the material to be tested for the presence of a biological marker using the present methods includes inter alia tissue, e.g., from a biopsy or tumor resection. The type of sample used may vary depending upon the clinical situation in which the method is used. Various methods are well known within the art for the identification and/or isolation and/or purification of a biological marker (e.g., IRF2, TAP2, or ERAP1, or PD-L1) from a sample. An “isolated” or “purified” biological marker is substantially free of cellular material or other contaminants from the cell or tissue source from which the biological marker is derived, i.e. partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

The presence and/or level of a protein can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84 (14):6008-6015; Brody (2010) Expert Rev Mol Diagn 10 (8):1013-1022; Philips (2014) PLOS One 9 (3):e90226; Pfaffe (2011) Clin Chem 57 (5): 675-687). The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance. Antibodies to IRF2, TAP2, and ERAP1 are known in the art, and are commercially available, e.g., from Abbexa Ltd; Abcam; Abnova Corporation; antibodies-online; AssayPro; Atlas Antibodies; Bioassay Technology Laboratory; BioLegend; Biorbyt; Bioss Inc.; Bioworld Technology; BosterBio; Creative Biolabs; Creative Diagnostics; Developmental Studies Hybridoma Bank/DSHB; Fitzgerald Industries International; GeneTex; HuaBio; Invitrogen Antibodies; LifeSpan BioSciences; MilliporeSigma; MyBioSource.com; Novus Biologicals; NSJ Bioreagents; OriGene Technologies; ProSci, Inc; Proteintech Group Inc; R&D Systems; RayBiotech; Signalway Antibody LLC; United States Biological; and Wuhan Fine Biotech Co., Ltd.

In some embodiments, an ELISA method may be used, wherein the wells of a mictrotiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

In some embodiments, an IHC method may be used. IHC provides a method of detecting a biological marker in situ. The presence and exact cellular location of the biological marker can be detected. Typically a sample is fixed with formalin or paraformaldehyde, embedded in paraffin, and cut into sections for staining and subsequent inspection by confocal microscopy. Current methods of IHC use either direct or indirect labelling. The sample may also be inspected by fluorescent microscopy when immunofluorescence (IF) is performed, as a variation to IHC.

Mass spectrometry, and particularly matrix-assisted laser desorption/ionization mass spectrometry (MALDI-MS) and surface-enhanced laser desorption/ionization mass spectrometry (SELDI-MS), is useful for the detection of biomarkers of this invention. (See U.S. Pat. No. 5,118,937; 5,045,694; 5,719,060; 6,225,047)

The presence and/or level of a nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48 (8): 1178-1185; Miranda (2010) Kidney International 78:191-199; Bianchi (2011) EMBO Mol Med 3:495-503; Taylor (2013) Front. Genet. 4:142; Yang (2014) PLOS One 9 (11):e110641); Nordstrom (2000) Biotechnol. Appl. Biochem. 31 (2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289 (5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of IRF2. Measurement of the level of a biomarker can be direct or indirect. For example, the abundance levels of IRF2, TAP2, or ERAP1, or PD-L1 can be directly quantitated. Alternatively, the amount of a biomarker can be determined indirectly by measuring abundance levels of cDNA, amplified RNAs or DNAs, or by measuring quantities or activities of RNAs, or other molecules that are indicative of the expression level of the biomarker. In some embodiments a technique suitable for the detection of alterations in the structure or sequence of nucleic acids, such as the presence of deletions, amplifications, or substitutions, can be used for the detection of biomarkers of this invention.

RT-PCR can be used to determine the expression profiles of biomarkers (U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes are most commonly used.

Gene arrays are prepared by selecting probes that comprise a polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

In some embodiments, once it has been determined that a subject has a level of IRF2, TAP2, or ERAP1, or PD-L1 above a reference, then a treatment comprising a checkpoint inhibitor, e.g., as known in the art or as described herein, can be selected and optionally administered. In some embodiments, once it has been determined that a subject has a level of IRF2, TAP2, or ERAP1, or PD-L1 below a reference, then a treatment comprising an agent that increases levels of interferons (e.g., interferon itself) and a checkpoint inhibitor, e.g., as known in the art or as described herein, can be selected and optionally administered. Alternatively, for a subject who has a level of IRF2, TAP2, or ERAP1, or PD-L1 below a reference level, a treatment that does not include a checkpoint inhibitor can be selected and optionally administered, e.g., a standard treatment comprising chemotherapy, radiotherapy, and/or resection. These standard treatments can also be administered in combination with a checkpoint inhibitor for subjects with levels of IRF2, TAP2, or ERAP1, or PD-L1 above the reference, or in combination with an agent that increases levels of interferons and a checkpoint inhibitor for subjects with levels of IRF2, TAP2, or ERAP1, or PD-L1 below the reference.

Suitable reference values can be determined using methods known in the art, e.g., using standard clinical trial methodology and statistical analysis. The reference values can have any relevant form. In some cases, the reference comprises a predetermined value for a meaningful level of IRF, TAP2, or ERAP12, or PD-L1, e.g., a reference level that represents a threshold level of IRF2, TAP2, or ERAP1, or PD-L1, e.g., above which the subject is considered likely to respond to a checkpoint inhibitor, and below which the subject is considered unlikely to respond to a checkpoint inhibitor absent administration of an agent that increases levels of interferons (or other treatment that would overcome the loss of IRF2, TAP2, or ERAP1).

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

The predetermined value can depend upon the particular population of subjects (e.g., human subjects) selected. For example, an apparently healthy population will have a different ‘normal’ range of levels of IRF2, TAP2, or ERAP1, or PD-L1 than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

In some embodiments, treatment selection is made based on whether IRF2 (or TAP2 or ERAP1) levels are above or below reference. If IRF2 (or TAP2 or ERAP1) levels are above the reference range (and express PD-L1, i.e., have PD-L1 levels above a reference) then PD-L1 checkpoint blockade is chosen and optionally administered. If IRF2 (or TAP2 or ERAP1) levels are above the reference range and PDL-1 is very low, then, a non-PD-L1 checkpoint blockade would be chosen. If IRF2 (or TAP2 or ERAP1) levels are below the reference range, and PD-L1 is above the reference, then PD-L1 with an IRF2 inducer (such as IFN) would be used.

### Methods of Treatment

The methods described herein include methods for the treatment of disorders associated with abnormal apoptotic or differentiative processes, e.g., cellular proliferative disorders or cellular differentiative disorders, e.g., cancer, including both solid tumors and hematopoietic cancers. In some embodiments, the disorder is a solid tumor, e.g., breast, prostate, pancreatic, brain, hepatic, lung, kidney, skin, or colon cancer. Generally, the methods include administering a therapeutically effective amount of a treatment as described herein, to a subject who is in need of, or who has been determined to be in need of, such treatment. In some embodiments, the methods include administering a therapeutically effective amount of a treatment comprising a checkpoint inhibitor, a treatment comprising an agent that increases levels of interferons and a checkpoint inhibitor, and/or a standard treatment comprising chemotherapy, radiotherapy, and/or resection. These standard treatments can also be administered in combination with a checkpoint inhibitor for subjects with levels of IRF2, TAP2, or ERAP1 above the reference, or in combination with (i) an agent that increases levels of interferons and/or an epigenetic modifier and (ii) a checkpoint inhibitor for subjects with levels of IRF2, TAP2, or ERAP1 below the reference.

As used in this context, to “treat” means to ameliorate at least one symptom of the disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, inter alia.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin. As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CIVIL) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

### Checkpoint Inhibitors

Immune checkpoint blockade has shown remarkable results in certain cancers and patient groups; currently approved immune checkpoint blockers are monoclonocal antibodies (mAbs) that target the programmed cell death protein 1 (PD-1)/PD-L1/2 or cytotoxic T-lymphocyte-associated protein 4 (CTLA-4) pathways, and agents targeting other pathways are in clinical development (including OX40, Tim-3, and LAG-3) (See, e.g., Leach et al., Science 271, 1734-1736 (1996); Pardoll, Nat. Rev. Cancer 12, 252-264 (2012); Topalian et al., Cancer Cell 27, 450-461 (2015); Mahoney et al., Nat Rev Drug Discov 14, 561-584 (2015)). The present methods can include the administration of checkpoint inhibitors such as antibodies including anti-CD137 (BMS-663513); anti-PD-1 (programmed cell death 1) antibodies (including those described in U.S. Pat. Nos. 8,008,449; 9,073,994; and US20110271358, pembrolizumab, nivolumab, Pidilizumab (CT-011), BGB-A317, MEDI0680, BMS-936558 (ONO-4538)); anti-PDL1 (programmed cell death ligand 1) or anti-PDL2 (e.g., BMS-936559, MPDL3280A, atezolizumab, avelumab and durvalumab); or anti-CTLA-4 (e.g., ipilumimab or tremelimumab). See, e.g., Krüger et al., “Immune based therapies in cancer,” Histol Histopathol. 2007 June; 22 (6):687-96; Eggermont et al., “Anti-CTLA-4 antibody adjuvant therapy in melanoma,” Semin Oncol. 2010 October; 37 (5):455-9; Klinke D J 2nd, “A multiscale systems perspective on cancer, immunotherapy, and Interleukin-12,” Mol Cancer. 2010 Sep. 15; 9:242; Alexandrescu et al., “Immunotherapy for melanoma: current status and perspectives,” J Immunother. 2010 July-August; 33 (6):570-90; Moschella et al., “Combination strategies for enhancing the efficacy of immunotherapy in cancer patients,” Ann N Y Acad Sci. 2010 April; 1194:169-78; Ganesan and Bakhshi, “Systemic therapy for melanoma,” Natl Med J India. 2010 January-February; 23 (1):21-7; Golovina and Vonderheide, “Regulatory T cells: overcoming suppression of T-cell immunity.” Cancer J. 2010 July-August; 16 (4):342-7.

In addition to or as an alternative to checkpoint inhibitors, the present methods can be used to predict benefit from and select treatment with any type of immunotherapy whose mechanism is CD8 T cell-mediated (e.g., vaccines, dendritic cell-based immunizations, or adoptively transferred anti-tumor CD8 T cells, etc); see, e.g., Durgeau et al., Front Immunol. 2018; 9: 14, doi: 10.3389/fimmu.2018.00014.

### Interferon Inducers

A number of agents are known in the art that are interferon inducers, i.e., that promote the production and release of interferons and thereby increase levels of interferons. These agents include type I or type 2 interferons themselves (e.g., interferon alpha-2b, PEGylated interferon alpha-2b, PEGylated interferon-alpha-2a, Human leukocyte Interferon-alpha (HuIFN-alpha-Le), Interferon beta 1a, Interferon beta 1b, Interferon gamma 1b), or other inducers of interferons (e.g., poly(I:C), Poly(A:U), ampligen [poly(I)-poly(Cl2U)], polyICLC), Imiquimod, and other TLR-3 agonists. See, e.g., Ammi et al., Pharmacol Ther. 2015 February; 146:120-31; Makita et al., Int J Oncol. 2019 May; 54 (5):1864-1874; and Urosevic and Dummer, Am J Clin Dermatol. 2004; 5 (6):453-8; Lee and Ashkar, Front Immunol. 2018; 9: 2061.

### Epigenetic Modifiers

In some embodiments, the present methods include administering an epigenetic modifier, e.g., a hypomethylating agent (such as a DNMT1 inhibitor) or a Histone deacetylase (HDAC) inhibitor.

### HDAC Inhibitors

In some embodiments, the methods include administration of an HDAC inhibitor, a number of which are known in the art, including Suberoylanilide hydroxamic acid (SAHA/Vorinostat/Zolinza), Trichostatin A (TSA), and belinostat (PXD101) (hydroxamic acid-based pan-HDAC inhibitors); depsipeptide (FK228/romidepsin/ISTODAX®) (a natural cyclic peptide inhibitor of HDAC1/2); Entinostat (SNDX-275; formerly MS-275) and mocetinostat (MGCD0103) (synthetic benzamide derivatives); valproic acid and Sodium phenylbutyrate (which are aliphatic acids with relatively low potency); LAQ824, panobinostat (LBH589), entinostat (MS275), CI-994 (N-acetyldinaline, also tacedinaline), EVP-0334, SRT501, CUDC-101, JNJ-26481585, PCI24781, and Givinostat (ITF2357). See, e.g., Kim and Bae, Am J Transl Res. 2011 Jan. 1; 3 (2): 166-179.

### DNA Hypomethylating Agents

In some embodiments, the methods described herein further comprise administering a DNA hypomethylating agent to a subject. DNA methylation is an epigenetic modification that regulates the silencing of gene transcription. In some embodiments, the DNA hypomethylating agent is a DNA methyltransferase (DNMT) inhibitor. In some embodiments, the DNA methyltransferase inhibitor is 5′-azacytidine (Aza), Decitabine (an FDA-approved cytosine analogue);
Cladribine/Fludarabine/Clofarabine (FDA-approved adenosine analogues that inhibit DNMT1 catalytic activity); Procainamide/Procaine (FDA-approved non-nucleoside inhibitors of DNMT1 catalytic activity), Zebularine (1-(β-D-ribofuranosyl)-1,2-dihydropyrimidin-2-one), (−)-epigallocatechin-3-gallate, MG98, hydralazine, RG108, and Chlorogenic acid, or a combination thereof (Wyczechowska et al. (2003) Biochem Pharmacol. 65: 219-25; Yu et al. (2006) Am. J. Hematol. 81 (11): 864-9; and Garcia-Manero (2008) Curr. Opin. Oncol. 20 (6): 705-10). Additional DNA hypomethylating agents are described, for example in U.S. Publication Nos. 2011/0218170A1, 2005/0119201, and 2015/0258068, the entire contents of each of which are incorporated herein by reference.

### EXAMPLES

The invention is further described in the following examples, which do not limit the scope of the invention described in the claims.

### Methods

The following materials and methods were used in the examples below.

### Cells

DC3.2 is a J2 virus-immortalized dendritic cell line¹³. A particular DC3.2 clone (with Renilla luciferase) was used for all experiments in this study as this clone has very strong cross-presentation and MHC class II presentation, as compared to other clones. RF33.70 is a T cell hybridoma that recognizes the ovalbumin (OVA) peptide OVA₂₅₇₋₂₆₄ in the context of H2-K^{b 46}. MF2.2D9 is a T cell hybridoma that recognizes OVA₂₅₈₋₂₇₆ in the context of I-A^{b 13}. RF33.70 and MF2.2D9 were transduced with lentivirus containing NFAT-luciferase. NIH-3T3 cells were stably transfected with the mouse H2-K^{b} molecule. A549 and MCF7 were kindly provided by Leslie Shaw (UMass), and the D53m and H50m mouse MCA-induced sarcoma lines were kindly provided by Robert Schreiber (Wash U, St. Louis). The MCA-induced sarcoma lines were grown in R10 media and all other cell lines were grown in RPMI 1640 (Gibco) supplemented with 10% FBS (Hyclone), 1% NEAA (Gibco), 1% HEPES (Gibco), 1% Antibiotic-Antimycotic (Gibco), and 5×10⁻⁵ M 2-ME (Sigma). The MCF7 growth media was also supplemented with 10 μg/mL insulin. Antibiotic selection for CRISPR-Cas9 knockout cells was done for two weeks in media containing 5 μg/mL blasticidin (Invivogen). All cells were grown in a 10% CO₂ atmosphere at 37° C.

### Plasmids

The LentiCRISPRv2 plus blasticidin selection plasmid^{47,48} was acquired from Addgene (83480) and, unmodified, is the same as the “no sgRNA” plasmid. The plasmids used to target mouse B2m, TAP1, TAP2, ERAP1, IRF2, IRF1 or to target human IRF2 were constructed by inserting the following sgRNA sequences, respectively, into the LentiCRISPRv2 plasmid as described below:

Mouse B2m:
(SEQ ID NO: 1)
5′-AGTATACTCACGCCACCCAC-3′;
mouse TAP1:
(SEQ ID NO: 2)
5′-ACTAATGGACTCGCACACGT-3′;
mouse TAP2:
(SEQ ID NO: 3)
5′-ATTACACGACCCGAATAGCG-3′;
mouse ERAP1:
(SEQ ID NO: 4)
5′-TGCAGCATCCAGAGCATAAT-3′;
mouse IRF2:
(SEQ ID NO: 5)
5′-TCCGAACGACCTTCCAAGAA-3′;
mouse IRF1:
(SEQ ID NO: 6)
5′-CTCATCCGCATTCGAGTGAT-3′;
human IRF2:
(SEQ ID NO: 7)
5′-TGCATGCGGCTAGACATGGG-3′.

Primer sets for cloning the sgRNAs above into the LentiCRISPRv2 plasmid were as follows:

| | | SEQ ID | | SEQ |
|---|---|---|---|---|
| | Forward (5′-3′) | NO: | Reverse (5′-3′) | ID NO: |
| Mouse | CACCGAGTATACTCACGCCACCCAC | 8 | AAACGTGGGTGGCGTGAGTATAC | 9 |
| B2m | | | TC | |
| Mouse | CACCGACTAATGGACTCGCACACGT | 10 | AAACACGTGTGCGAGTCCATTAG | 11 |
| TAP1 | | | TC | |
| Mouse | CACCGATTACACGACCCGAATAGCG | 12 | AAACCGCTATTCGGGTCGTGTAA | 13 |
| TAP2 | | | TC | |
| Mouse | CACCGTGCAGCATCCAGAGCATAAT | 14 | AAACATTATGCTCTGGATGCTGC | 15 |
| ERAP1 | | | AC | |
| Mouse | CACCGTCCGAACGACCTTCCAAGAA | 16 | AAACTTCTTGGAAGGTCGTTCGG | 17 |
| IRF2 | | | AC | |
| Mouse | CACCGCTCATCCGCATTCGAGTGAT | 18 | AAACATCACTCGAATGCGGATGA | 19 |
| IRF1 | | | GC | |
| Human | CACCGTGCATGCGGCTAGACATGGG | 20 | AAACCCCATGTCTAGCCGCATGC | 21 |
| IRF2 | | | AC | |

Construct cloning was done as follows: 100 μM oligonucleotides from the primer sets were annealed and then diluted 1:50. 3 μg of LentiCRISPRv2 plasmid was digested for 3 hrs at 55° C. with BsmBI (NEB) and removal of the 2 kb filler sequence was confirmed by gel electrophoresis. The larger molecular weight band was gel extracted and quick ligated with the diluted annealed oligos according to the manufacturer's instructions (NEB). Stable competent E. coli (NEB) were then transformed with 2 μL of the quick ligation product according to the manufacturer's instructions and grown overnight at 30° C. on LB+Ampicillin (100 μg/mL) plates. Plasmids were isolated (Clontech) from individual colonies and sequenced (Genewiz) using the primer hU6-F: 5′-GAGGGCCTATTTCCCATGATT-3′ (SEQ ID NO:22) to confirm proper insertion of the sgRNA into LentiCRISPRv2. In addition, sgRNAs were checked for high indel efficiencies in transduced cells by TIDE analysis¹⁴.

Rescue plasmids were constructed by inserting mouse IRF2, TAP2, or ERAP1 cDNA or human IRF2 cDNA into the constitutive expression vector, pCDH-CMV (Addgene), with a modified multiple cloning site. Overlapping PCRs were run using the primers in the table below on either mouse cDNA from DC3.2 cells or human cDNA from HeLa H1 cells to create IRF2 cDNA sequences containing 6 synonymous mutations within the IRF2 sgRNA target site. The mouse IRF2 K78R sequence was constructed by further mutating A to G at nucleotide 233. The wild-type TAP2 and ERAP1 cDNA sequences were of C57BL/6 origin. All plasmids were sequenced to confirm correct sequences and reading frames.

TABLE
Primer sets for cloning rescue/overexpression constructs

| Primer name | Sequence | SEQ ID NO: |
|---|---|---|
| Human IRF2 Agel | GACTACCGGTATGCCGGTGGAAAGGATGCGCATG | 23 |
| fwd | | |
| Human IRF2 sgRNA | GCCGTGCCTCGCTGCGTGCATCCAGGGGATCTGAA | 24 |
| nnut rev | AAATCTTCTTTTCCTTG | |
| Human IRF2 sgRNA | GATGCACGCAGCGAGGCACGGCTGGGATGTGGAA | 25 |
| mut fwd | AAAGATGCACCACTCTTTAGAAA | |
| Human IRF2 Mlul | GATCACGCGTTTAACAGCTCTTGACGCGGGCCTGG | 26 |
| rev | | |
| Mouse IRF2 Agel | GATCACCGGTATGCCGGTGGAACGGATGCGAATG | 27 |
| fwd | | |
| Mouse IRF2 sgRNA | CCTTTTTTCGAGGGGCGCTCTGATAAGGGCAGCAT | 28 |
| mut rev | CCGGTAGACTCTGAAGGCG | |
| Mouse IRF2 sgRNA | CTTATCAGAGCGCCCCTCGAAAAAAGGAAAGAAACC | 29 |
| mut fwd | AAAGACAGAAAAAGAAGAGAG | |
| Mouse IRF2 Mlul | GATCACGCGTTTAACAGCTCTTGACACGGGCCTGG | 30 |
| rev | | |

### Cell Surface Staining

Where indicated, mouse cells were blocked with 2.4G2 and stained for surface MHC class I levels with anti-K^{b}-APC (eBioscience, AF6-88.5.5.3), MHC class II levels with anti-I^{A}/I^{E}-PECy7 (BioLegend, M5/114.15.2), PD-L1 levels with anti-PD-L1-PE (BioLegend, 10F.9G2), or with isotype controls (eBioscience mouse IgG2a-APC eBM2a, eBioscience rat IgG2b κ-PE eB149) at 1:200 dilutions. Where indicated, human cells were stained for surface MHC class I levels with W6/32. W6/32 staining was performed either by two-step labeling with W6/32 hybridoma supernatant followed by 1:500 donkey-anti-mouse Alexa 647 (Life Technologies) or by one-step labeling with 1:200 FITC-conjugated W6/32 (eBioscience). Where indicated, human cells were stained for surface PD-L1 levels with 1:200 rabbit anti-PD-L1 (Abcam, 28-8), followed by 1:500 donkey-anti-rabbit Alexa 647 (Life Technologies). Normalized MFI was computed by dividing the geometric MFI of each knockout cell line by the geometric MFI of the WT (no sgRNA) cell line.

### T Cell Hybridoma Ag Presentation

Cross-presentation and MHC class II presentation were measured by co-culturing DC3.2 lines in the presence of the indicated concentrations of OVA-coated iron-oxide beads (Polysciences) and RF33.70-Luc CD8⁺ T cells or MF2.2D9-Luc CD4⁺ T cells, respectively, for 16-18 hours. Then, One-Glo luciferase substrate (Promega) was added and luciferase activity quantified by an EnVision plate reader (Perkin Elmer). Rescue experiments were performed by adding the OVA-beads and RF33.70-Luc cells 48 hrs post-transduction of the DC3.2 lines. Data from a representative cross-presentation and MHC class II presentation experiment (of N=4) are shown where points represent mean±SD of technical duplicates. Normalized CD8⁺ T cell activation was calculated for rescue experiments by dividing the CD8⁺ T cell activation (RLU of luciferase) at each point by the CD8⁺ T cell activation of the DC3.2 no sgRNA line transduced with EV (WT+EV).

### siRNA Transfections

10⁴ HEK293T or HeLa H1 cells were transfected with 10 nM Silencer Select siRNA (Invitrogen) and 0.3 μL Lipofectamine RNAiMAX (Invitrogen) per well in flat-bottom 96-well plates. Individual siRNAs used were negative control #1 (4390843) human β32m (s1854), human TAP1 (s13778), human IRF2 #1 (s7506), and human IRF2 #2 (s7504). After 72 hours, adherent cells were trypsinized, washed in PBS supplemented with 2% FBS, and stained with the surface MHC I pan-HLA-A/B/C antibody, W6/32. Normalized MFI was computed by dividing the geometric MFI of each experimental siRNA by the geometric MFI of the negative control siRNA.

### Minigene Transfections

10⁴ 3T3-Kb cells were transfected with 100 ng of various pTracer-CMV2 plasmids (Invitrogen) containing SIINFEKL precursors⁴⁹ and 0.4 uL Lipofectamine 2000 (Invitrogen) in flat-bottom 96-well plates. After 72 hours, cells were stained with 25-D1.16 (specific for the combination of SIINFEKL and H-2K^{b})¹⁶, followed by donkey-anti-mouse Alexa 647 (Life Technologies) and analyzed by flow cytometry. Transfected cells were identified by GFP expression and the MFI of 25-D1.16 staining was measured on the gated transfected cells. Transfection efficiency, based on GFP expression, ranged from 5-20%, depending on the vector. The normalized MFI for each experiment (with technical duplicates) was calculated by dividing the MFI of each knockout line by the MFI of the wild-type (“no sgRNA”) line.

### RNA-Seq

RNA was extracted using the RNeasy kit (Qiagen) after 2 hours of stimulating the DC3.2 lines with 5,000 U/mL mouse IFNα, 2 ng/mL mouse IFNγ, or media alone. A standard library preparation protocol was used with 50 ng of total RNA as starting material. Libraries were checked for appropriate fragment size traces by Bioanalyzer (Agilent) and concentrations were determined to achieve similar sequencing depth per library. Libraries were run on NextSeq 500/550 high-output and mid-output kits (Illumina) and all libraries had at least 10⁷ reads with single index paired-end sequencing. Trimmomatic-0.32⁵⁰ was used to remove 5′ or 3′ stretches of bases having an average quality of less than 20 in a window size of 10. Only reads longer than 36 bases were kept for further analysis. RSEM v1.2.28⁵¹ was used to estimate gene expression, with parameters-p 4—bowtie-e 70—bowtie-chunkmbs 100—strand-specific. Gene quantification was run on the transcriptome (RefSeq v69 downloaded from UCSC Table Browser⁵². Genes with more than 15 TPM in any time point were considered expressed, and genes that did not achieve this threshold were removed from further analysis. Batch effects were observed between samples from different replicates. We used the log transformed TPM normalized expression values as input to ComBat (package sva version 3.18.0)^{53,54} with default parameters and a model that specified different replicates as batches. Corrected TPM values were transformed back to read counts using the expected size of each transcript informed by RSEM. We only considered genes with at least 15 TPMs in at least one replicate at any time point. The expressed gene list was filtered to include only genes with homologs as defined by the previous step. We used the batch corrected counts per gene to identify differentially expressed genes by at least 2 fold between unstimulated cells (time 0) and 2 hours following stimulation with IFNα or IFNγ and whose change in expression was significant (p-adjusted <0.05) according to the package DESeq2 (v1.10.1)⁵⁵ in R (v3.5.1). Due to the large transcriptional changes observed in this system, we turned off the fold change shrinkage in DESeq2 with betaPrior=FALSE and we added a pseudocount of 32 to all timepoints to avoid spurious large fold change estimates from lowly abundant genes.

### Human Lung Specimens

27 non-small cell lung cancers were acquired from UMass Pathology archive of formalin-fixed and paraffin-embedded patient samples. PD-L1 expression was determined at the time of diagnosis by immunohistochemistry (22C3 pharmDx assay; Agilent) by a surgical pathologist. RNA was extracted from the archival material and analyzed by RT-PCR (see below).

### RT-PCR

RNA was extracted from cell lines using the RNeasy kit (Qiagen) or from formalin-fixed paraffin-embedded non-small cell lung cancers using the RNeasy FFPE kit (Qiagen) and reverse transcribed to cDNA using EcoDry pre-mix random hexamers (Clontech). 50 ng cDNA were used per well (done in triplicate) with indicated TaqMan probes (Applied Biosystems), according to the manufacturer's instructions. The TaqMan probes used were as follows:

| Gene | Probe ID # |
|---|---|
| Mouse β-actin | 4352933 |
| Mouse β2m | Mm00437762_m1 |
| Mouse H2-K1 | Mm01612247_m1 |
| Mouse H2-Ab1 | Mm00439216_m1 |
| Mouse Tap1 | Mm00443188_m1 |
| Mouse Tap2 | Mm01277033_m1 |
| Mouse Erap1 | Mm00472842_m1 |
| Mouse ERp57 | Mm00433130_m1 |
| Mouse Tapasin | Mm00493417_m1 |
| Mouse Canx | Mm00500330_m1 |
| Mouse Calr | Mm00482936_m1 |
| Mouse Tapbpr | Mm00520408_m1 |
| Mouse Irap | Mm00555903_m1 |
| Mouse Psme1 | Mm00650858_g1 |
| Mouse Psme2 | Mm01702833_g1 |
| Mouse Psmb8 | Mm00440207_m1 |
| Mouse Psmb9 | Mm00479004_m1 |
| Mouse Psmb10 | Mm00479052_g1 |
| Mouse PD-L1 | Mm03048248_m1 |
| Human GAPDH | Hs02758991_g1 |
| Human IRF2 | Hs01082884_m1 |
| Human TAP2 | Hs00241060_m1 |
| Human ERAP1 | Hs00429970_m1 |

mRNA expression levels in the IRF2-knockout DC3.2 were compared to those in the wild-type DC3.2 by first normalizing to the mRNA expression of (3-actin (mouse) in each sample (2\hat{ }^{-ΔΔCt}). Statistical analysis by comparing the expression of a given gene to that of H2-Ab 1. Relative mRNA expression levels in each human lung tumor were measured by first normalizing to the mRNA expression of GAPDH (human) in each specimen (2\hat{ }^{-ΔCt}). Results are displayed after further normalization to one of the tumors. Statistical analysis was done using two-tailed Mann-Whitney U-tests and linear regression models with R² for goodness of fit.

### Chromatin Immunoprecipitations

ChIP procedure generally followed the Thermo Fisher ChIP protocol. In short, 10⁷ DC3.2-no sgRNA cells were stimulated for 2 hrs with 2 ng/mL IFNγ or media alone, harvested and fixed with 1% formaldehyde, quenched with glycine, and washed in cell lysis buffer then nuclear lysis buffer. Chromatin was sheared by sonication for 20 min and fragment size (˜300 bp) was determined by gel electrophoresis. Immunoprecipitations of the sheared chromatin were done using 2 μg of primary antibody—normal rabbit IgG (Santa Cruz sc-2027), rabbit anti-IRF1 (Abcam ab186384), or rabbit anti-IRF2 (Invitrogen B-80 H53L46)—by incubating for 1 hr at RT then overnight at 4° C. The next day, 25 uL of pre-washed Protein A/G beads (Pierce) were added to each of the samples and incubated at RT for 30 min then 90 min at 4° C. After washing sequentially with low-salt buffer, high-salt buffer, LiCl buffer, and TE buffer, the DNA was eluted from the beads. All immunoprecipitated samples were treated with RNase A (Qiagen) and Proteinase K (Qiagen) and then column purified (Clontech). ChIP-qPCR was performed in triplicate wells using SYBR Green (Bio Rad) and unique primer sets (Table below) flanking the IRF1/2-binding site within the gene's promoter⁵⁶⁻⁵⁸, according to the manufacturer's instructions. Data shown as fold enrichment (2\hat{ }^{-ΔCt}) over the normal rabbit IgG control IP (N=2).

TABLE
Primer sets for ChIP-qPCR

| | | SEQ ID | | SEQ |
|---|---|---|---|---|
| | | | | ID |
| Gene | Forward primer | NO: | Reverse primer | NO: |
| Mouse | CAAATTGACAGGCG | 31 | GCTTCTTCTCAAAC | 32 |
| TAP2 | CCATCT | | TGGATCTCC | |
| Mouse | CTTAGGCTTGCTCT | 33 | GACTCCTGCTCCCG | 34 |
| ERAP1 | CTTTTAGCG | | ATCCTC | |
| Mouse | CAAGAAAGCTAATG | 35 | CCTGCGGATGACTT | 36 |
| PD-L1 | CAGGTTTCAC | | TAGAGTC | |

### Western Blotting

Whole cell lysates were prepared in RIPA buffer with protease inhibitor (Pierce), protein concentrations were determined by BCA assay (Pierce), and 10 μg of denatured samples were run on 10% reducing gels (Genscript). After transfer, PVDF membranes (Millipore) were blocked with TBS-Tween 1×+5% milk and then blotted with rabbit anti-IRF2 (Abcam ab124744) or rabbit anti-IRF1 (Abcam ab186384) in TBS-Tween 1×+2% milk overnight at 4° C. The following day, membranes were washed 3× with TBS-Tween 1×, goat-anti-rabbit HRP (Millipore) was added for 1 hr at RT, membranes were washed 3×, and HRP substrate (Millipore) was added. Following exposure, membranes were stripped (Millipore), blocked, and re-blotted with mouse anti-β-actin (Santa Cruz sc-47778) in TBS-Tween 1×+2% milk overnight at 4° C. The following day, membranes were prepared as above except anti-mouse HRP (Pierce) was used instead.

### In Vitro Cytotoxicity Assays

OT-I CD8⁺ T cells were pre-activated by co-culturing them for 4-5 days with irradiated, SIINFEKL-pulsed wild-type LPS-stimulated B cell blasts in T cell media containing 30 ng/mL IL-2. After this time, OT-I were checked for CD8 expression and upregulation of CD27 and CD44. WT or IRF2-null RMA cells were counted, sub-divided into respective tubes, SIINFEKL-pulsed at the indicated concentrations or kept un-pulsed, and CFSE-labeled as high (1 μM) for cells pulsed with SIINFEKL and CFSE-labeled as low (0.1 μM) for un-pulsed cells. Pulsed and un-pulsed cells were re-counted, mixed 1:1, and 10⁵ cells total were plated per well in U-bottom 96-well plates. 5×10⁴ OT-I were added to the respective wells and incubated at 37° C. for 4 hrs. Live RMA cells were gated by flow cytometry and CFSE levels analyzed. Specific killing was determined for each RMA line by calculating:

$$100 \times [ 1 - \frac{( \frac{\% Targets}{\% Bystanders} )}{\frac{\% Control Targets}{\% Control Bystanders}} ] .$$

### Example 1

### IRF2 Positively Regulates the MHC-I Presentation Pathway

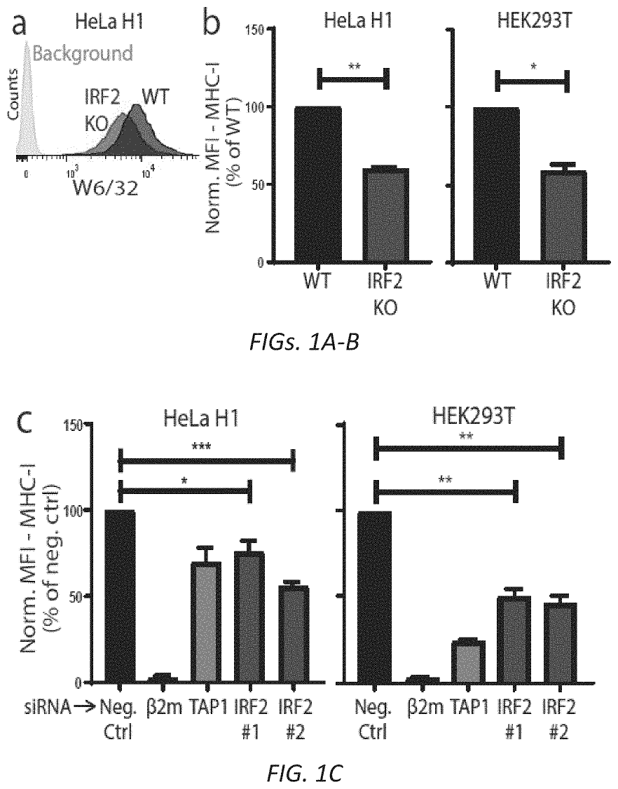

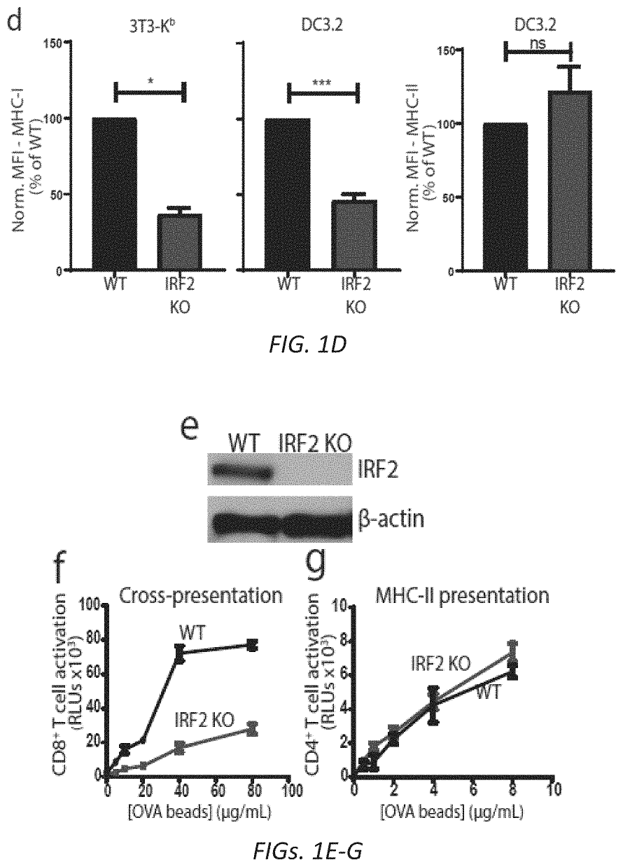

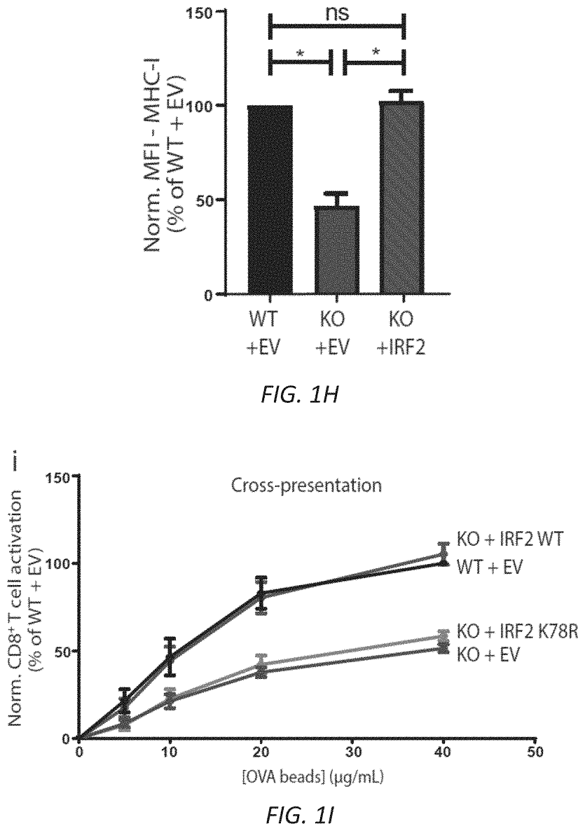

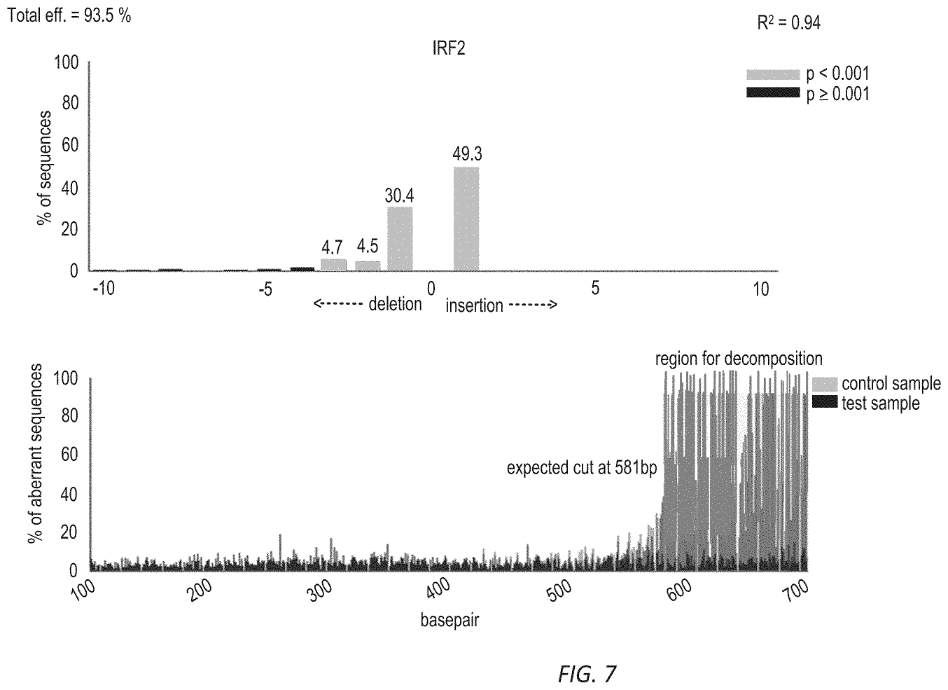

When HeLa H1 cervical carcinoma cells were immunoselected for MHC-I low variants in a genome-wide CRISPR-Cas9 screen, IRF2 scored as the second most targeted gene, with six out of six independent IRF2 guide RNAs hitting. To validate this hit, we tested whether IRF2 affects surface MHC-I levels in human cells by knocking out IRF2. HeLa H1 cells and HEK293T kidney cells were transduced with vectors expressing either Cas9 and a sgRNA targeting human IRF2 or Cas9 alone and surface MHC-I levels were checked by flow cytometry (FIG. 1A). The IRF2-knockout HeLa H1 and HEK293T had significantly lower surface MHC-I levels than their wild-type controls (FIG. 1B). To further validate this finding with an independent technique, we silenced IRF2 expression with siRNAs and found that this also decreased surface MHC-I levels in HeLa H1 and HEK293T (FIG. 1C), confirming the phenotype. The magnitude of the decrease in MHC-I was similar to that observed when the expression of the TAP transporter was silenced (FIG. 1C). To determine whether IRF2 also affects surface MHC-I levels in mouse cells, we transduced NIH-3T3 fibroblasts stably transfected with H2-K^{b} (3T3-K^{b}) and DC3.2 dendritic cells¹³ with vectors expressing either Cas9 and a sgRNA targeting mouse IRF2 or Cas9 alone. The IRF2-knockout (IRF2-KO) mouse fibroblasts and DC cells also had significantly reduced surface MHC-I levels (FIG. 1D). Disruption of the IRF2 gene was validated by TIDE analysis¹⁴ (FIG. 7) and loss of IRF2 expression was confirmed by western blot (FIG. 1E). DC3.2 cells also express MHC-II molecules and we found no change in surface MHC-II levels in the IRF2-KO cells (FIG. 1d), which demonstrates that IRF2 is selectively affecting the MHC-I pathway; further evidence supporting this conclusion will be described below. To investigate the functional consequence of this reduction in MHC-I levels, we evaluated the importance of IRF2 for MHC-I cross-presentation (i.e., the presentation of peptides derived from exogenous antigen on MHC-I). The IRF2-KO DCs cross-presented more poorly than their wild-type controls (FIG. 1F), demonstrating that IRF2 positively regulates MHC-I antigen presentation. In the same experiments, MHC-II presentation was unaffected, again showing selectivity in IRF2 effects (FIG. 1G). Lastly, to confirm that IRF2 is responsible for these differences, we overexpressed IRF2 in the IRF2-KO DC3.2 line and found that it completely restored surface MHC-I levels (FIG. 1H) and the ability of these cells to cross-present antigen (FIG. 1I). Evidence that loss of IRF2 also compromises the MHC-I presentation of endogenous cellular antigens will be described below.

To determine whether IRF2 was exerting its function as a transcription factor, we introduced a lysine to arginine point mutation at position 78 which prevents acetylation at this site and thereby prevents IRF2 from binding its DNA target sequences¹⁵.

Overexpressing this mutant IRF2 did not restore function in the IRF2-KO DC3.2 cells (FIG. 1i). Therefore, IRF2 is necessary for optimal transcriptional regulation of MHC-I antigen presentation pathways.

### Example 2

### How Does IRF2 Regulate the MHC-I Pathway?

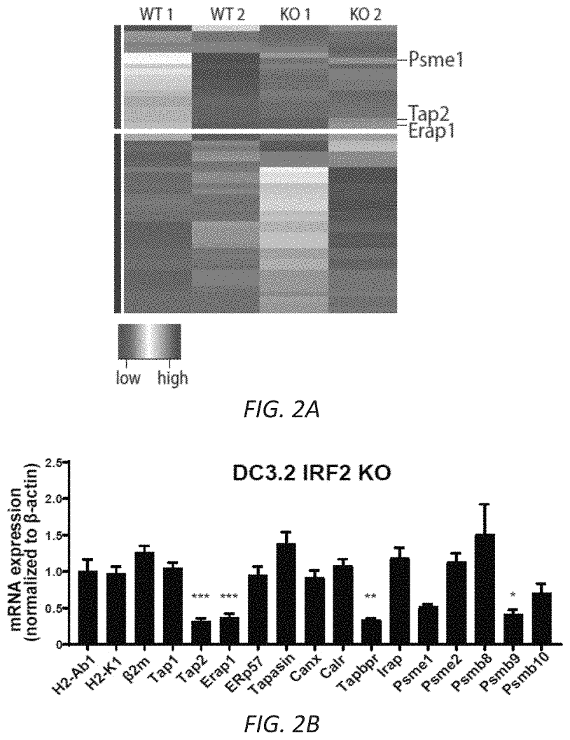

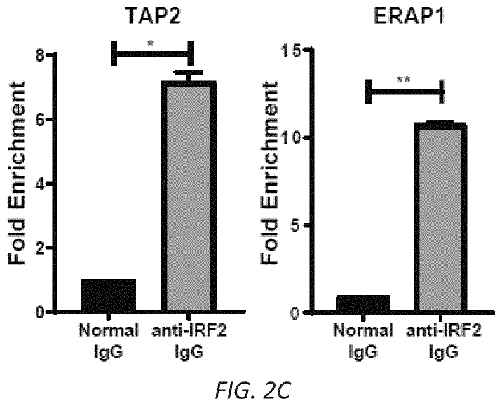

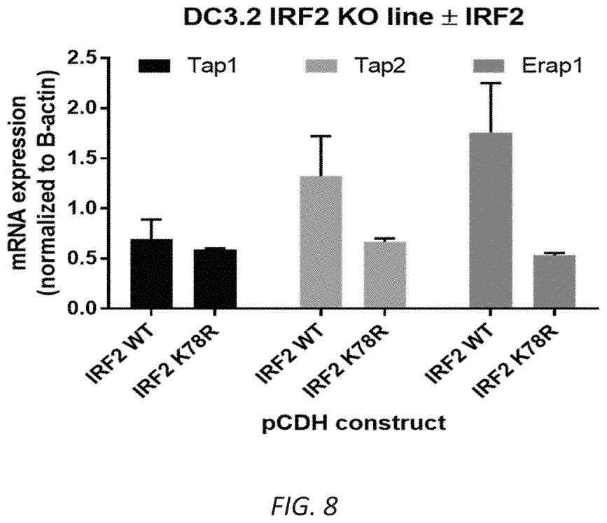

Since IRF2 is functioning as a transcription factor, we next sought to determine what genes are regulated by IRF2 and could be responsible for producing the low MHC-I phenotype observed in the IRF2-KO cells. RNA-seq was performed on the wild-type and IRF2-KO DC3.2 lines (FIG. 2A). Surprisingly, relatively few genes were differentially expressed by >2-fold (19 decreased and 33 increased). Of these 52 differentially expressed genes, we identified TAP2, ERAP1, and the immunoproteasome subunit PSME1 as potential contributors to the decreased MHC-I levels. To further confirm this result, we performed qPCR in these cell lines to check expression of all MHC-I pathway genes (FIG. 2B), which showed that the mRNA levels of TAP2, ERAP1, and PSME9 (another immunoproteasome subunit) were significantly downregulated in the IRF2-KO DC3.2; PSME1 levels were reduced but this decrease did not achieve statistical significance. Interestingly, the mRNA levels of the MHC heavy chain (H2-K1) and MHC light chain (Beta2-microglobulin; β32m) were unaffected (FIG. 2B), indicating that IRF2 is not required for synthesis of the MHC-I heterodimer. ChIP-qPCR experiments confirmed that IRF2 regulates TAP2 and ERAP1 mRNA expression by directly binding to their promoters (FIG. 2C). To test whether functional IRF2 is needed for TAP2 and ERAP1 mRNA expression, we overexpressed wild-type IRF2 or the IRF2-K78R mutant in the DC3.2 IRF2-KO cells and found that the TAP2 and ERAP1 mRNA levels were increased in the cells expressing wild-type but not mutant IRF2 (FIG. 8).

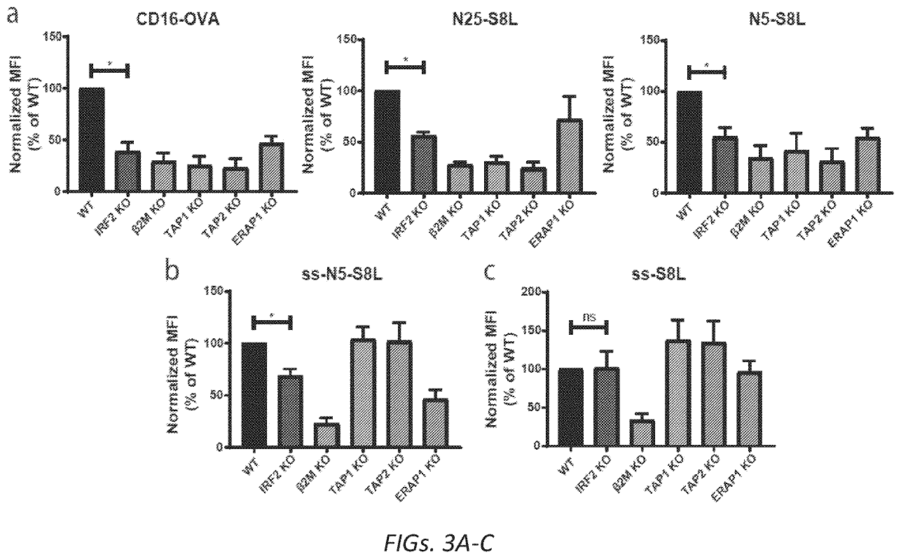

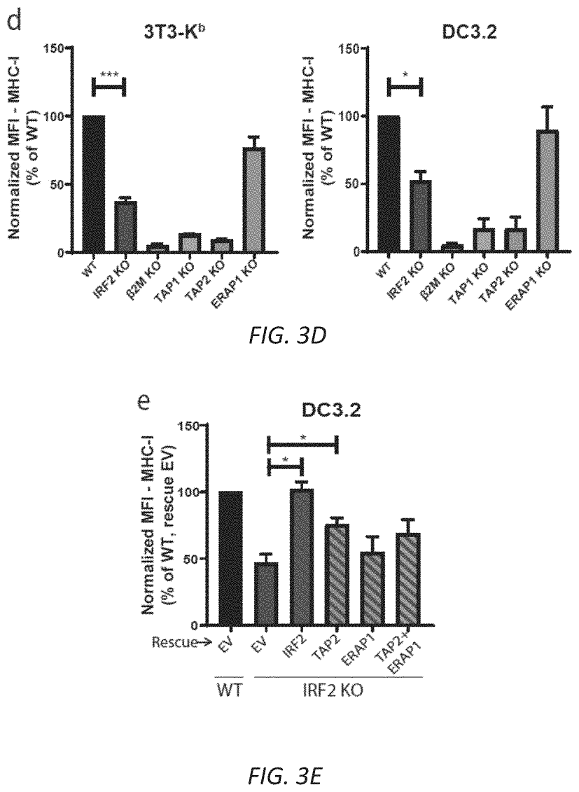

To more closely examine the functional effects of IRF2 on peptide transport and trimming, 3T3-K^{b} IRF2-KO cells were transfected with various pTracer plasmids, each of which contained a GFP reporter and a minigene encoding a peptide that could be processed down to the mature epitope (i.e., SIINFEKL/S8L). The cells were surface stained with the antibody 25-D1.16, which recognizes H2-K^{b}-S8L complexes¹⁶, and transfected cells (GFP-positive) were analyzed. IRF2-KO cells presented fewer H2-K^{b}-S8L complexes than wild-type cells when given the TAP-dependent, ERAP1-dependent antigens CD16-OVA (full-length ovalbumin protein)¹⁷, N25-S8L (a S8L precursor extended by 25 amino acids on the N-terminus), or N5-S8L (a S8L precursor extended by 5 amino acids on the N-terminus) (FIG. 3A). We also tested the presentation of a version of the precursor peptide with 5 extra N-terminal residues that was targeted into the ER by a co-linear signal sequence (ss-N5-S8L). Since the signal sequence allows this peptide to enter the ER through SEC61 instead of TAP, its presentation is TAP-independent but still dependent on ERAP1 to remove the extra N-terminal residues (FIG. 3b). IRF2-KO cells also presented fewer H2-K^{b}-S8L complexes than wild-type cells when transfected with ss-N5-S8L (FIG. 3B). However, IRF2-deficient cells were equally capable of presenting H2-K^{b}-S8L complexes when given a TAP-independent, ERAP1-independent peptide (S8L with no extra N-terminal residues that was targeted into the ER via a co-linear signal sequence; ss-S8L) (FIG. 3C), demonstrating that while IRF2 affects the transport and processing of MHC-I epitopes, it does not affect the ability of such peptides to be loaded onto MHC-I.

We also compared the MHC-I phenotype in IRF2-knockout 3T3-K^{b} to that observed in the same cells with knockouts of TAP2 or ERAP1. The magnitude of the reduction in MHC-I levels on IRF2-KO cells was in between that of the TAP2- and ERAP1-knockout cells (FIG. 3D), which is consistent with our findings that IRF2 positively regulates TAP2 and ERAP1 but their expression is not entirely lost in IRF2-KO cells. Lastly, we performed rescue experiments wherein we overexpressed TAP2 and/or ERAP1 in the IRF2-knockout DC3.2 and checked surface MHC-I levels two days after transduction (FIG. 3E). Although the double-rescue partially restored MHC-I levels, it was not complete, suggesting that other genes regulated by IRF2 also contribute to surface MHC-I expression.

### Example 3

### IRF2 and PD-L1

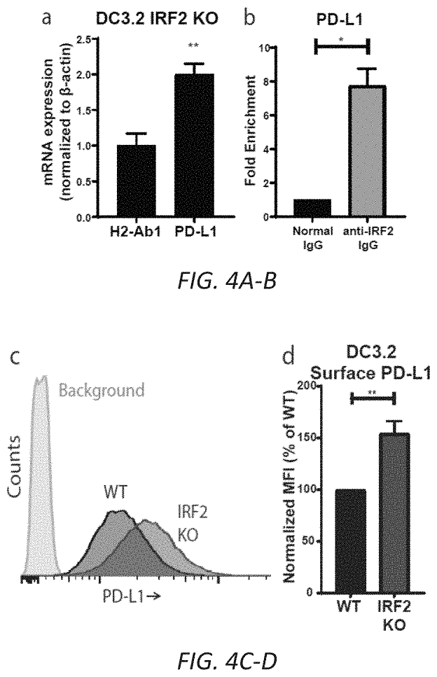

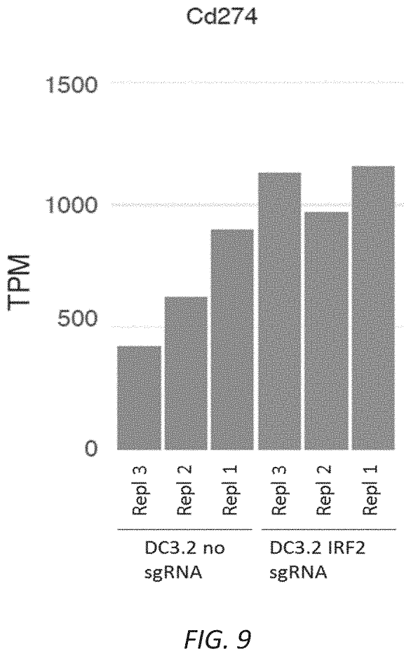

One of the upregulated genes in the IRF2-KO cells was Cd274 (FIG. 9), also known as programmed death-ligand 1 (PD-L1), which is often upregulated in certain cancers (e.g., non-small cell lung cancer) and functions as a checkpoint inhibitor to suppress antigen-specific CD8⁺ T cell effector function^{18,19}. To validate our RNA-seq finding with an independent technique, we performed qPCR on the IRF2-KO and wild-type DC3.2 lines and found that the IRF2-KO cells expressed approximately twice as much PD-L1 mRNA as the wild-type controls (FIG. 4A). Additionally, ChIP-qPCR revealed that IRF2 regulates PD-L1 mRNA expression by directly binding the PD-L1 promoter (FIG. 4B). Therefore, IRF2 acts as a transcriptional repressor of PD-L1 in these cells. To evaluate the extent to which a roughly 2-fold increase in PD-L1 mRNA translates to surface PD-L1 expression, we analyzed these cells by flow cytometry (FIG. 4C). This analysis reveals that the surface PD-L1 levels also increased by roughly 50% in the IRF2-KO cells (FIG. 4D). Taken together, these results demonstrate that IRF2 plays a role in repressing PD-L1 expression under basal conditions.

### Example 4

### IRF1-IRF2 Balance

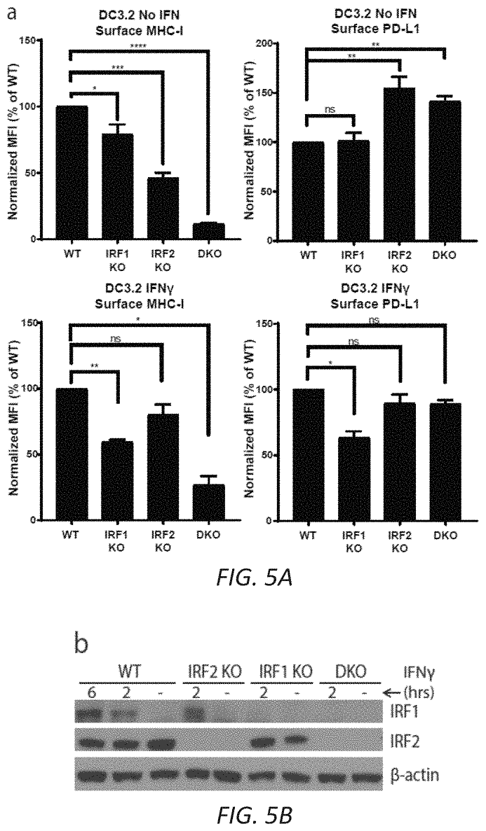

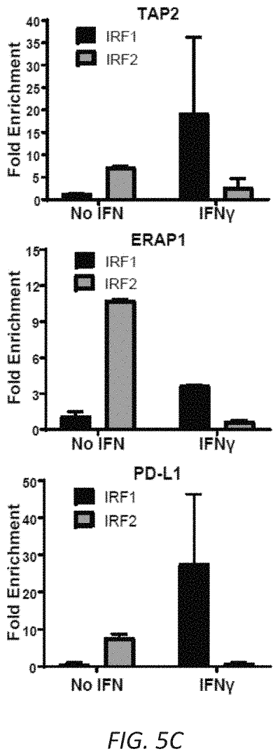

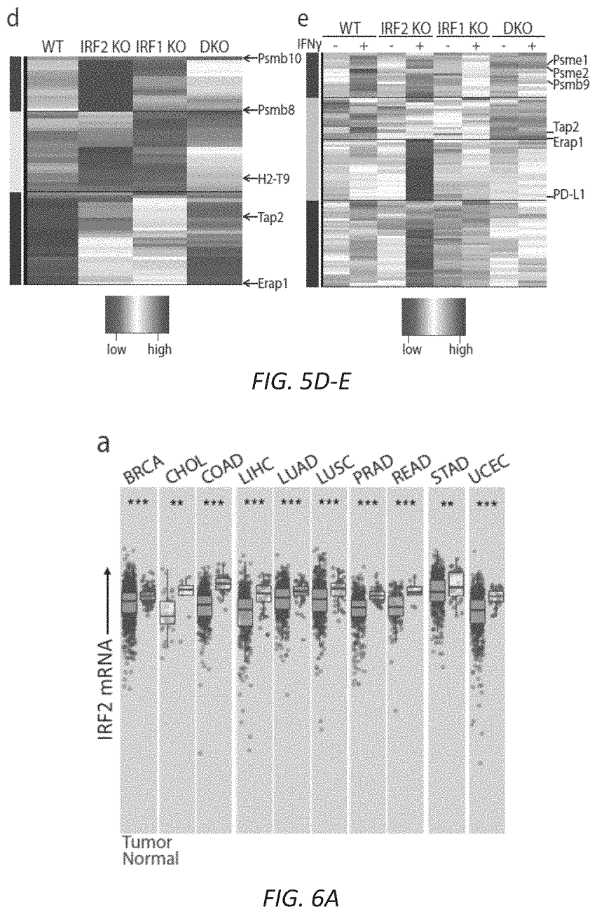

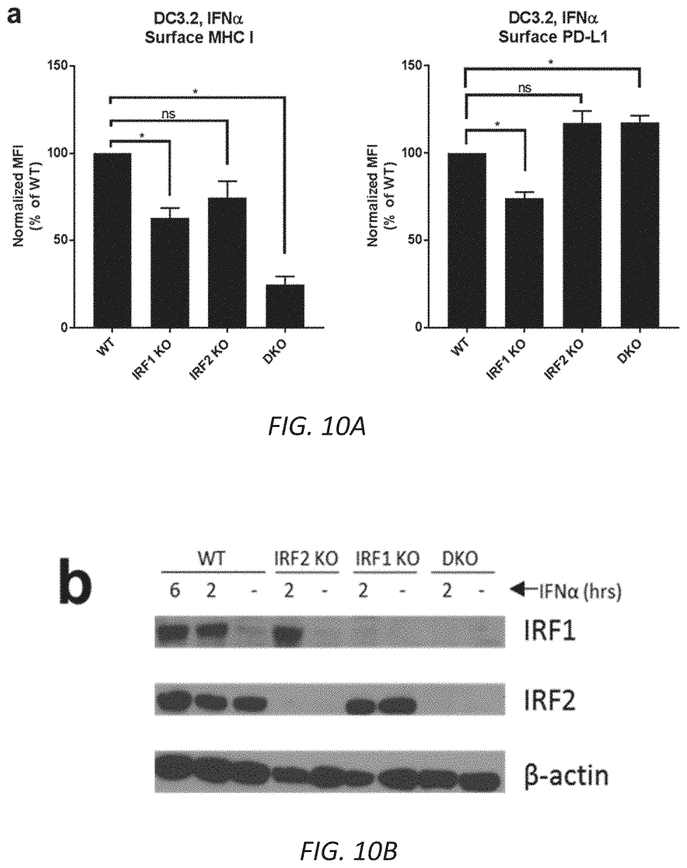

Because IRF2 is an interferon regulatory transcription factor, we wanted to see how interferon induction would affect IRF2′s regulation of the MHC-I pathway and PD-L1 expression. Interestingly, stimulation with either IFNγ or IFNα restored the surface MHC-I expression in the IRF2-KO DC3.2 (FIG. 5A, FIG. 10A-B). This is an important finding because it indicates that impairment of the MHC-I pathway from loss of IRF2 is reversible.

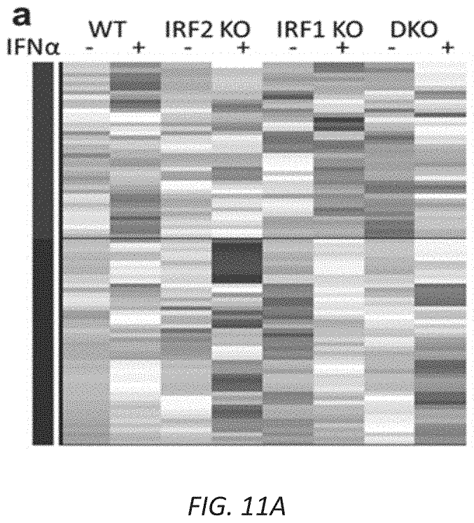

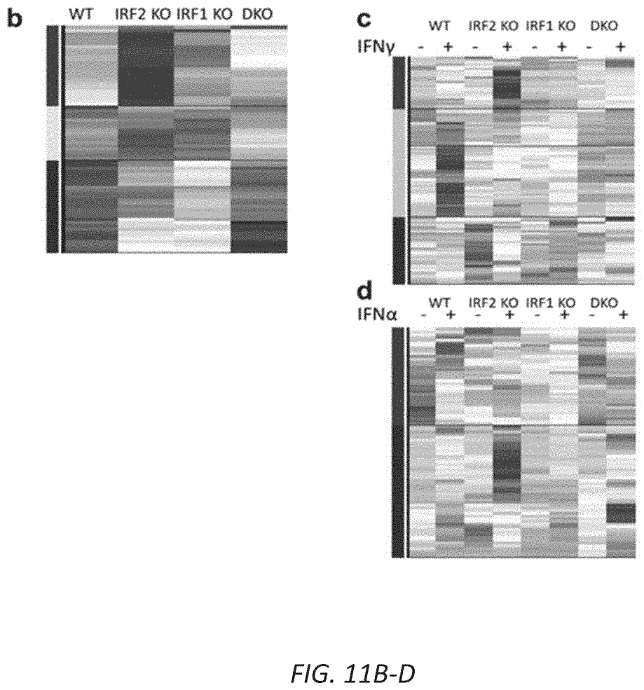

IRF1 and IRF2 recognize the same IFN-stimulated response element (ISRE)²⁰⁻²² and, whereas IRF2 is constitutively expressed and minimally affected by interferon induction, IRF1 is significantly upregulated in response to interferon (FIG. 5B, FIG. 10)²³⁻²⁵. Such upregulation of IRF1 causes IRF1 to compete with IRF2 for binding to the TAP2, ERAP1, and PD-L1 promoters, ultimately displacing it from them (FIG. 5C). Consistent with the literature that IRF1 positively regulates both MHC-I and PD-L1 expression under IFN-stimulated conditions²⁶⁻²⁸, we found that knocking out IRF1 decreased both surface MHC-I and PD-L1 levels after IFNγ stimulation (FIG. 5A). Interestingly, when we examined the dual effects of IRF1 and IRF2 on surface MHC-I and PD-L1 levels using single or double knockout DC3.2 lines (FIG. 5A, FIG. 10A-B), we found that: (1) IRF1/2 double knockouts have a larger reduction in surface MHC-I than is observed in either single knockout; and (2) IRF2-knockouts have a larger effect than the IRF1-knockouts on both MHC-I and PD-L1 expression under basal conditions. This suggested that, although these two IRFs recognize the same ISRE, the subset of genes they each primarily regulate differs and that a cell's dependence on IRF2 vs. IRF1 for any given gene may vary depending on the cues (e.g., interferon) which that cell receives from its environment. To better characterize these expression changes more globally, we performed RNA-seq on the single and double knockout DC3.2 lines under basal conditions (FIG. 5D) or after adding IFNγ (FIG. 5e) or IFNα (FIG. 11A-D). From this analysis, several genes known to be important for antigen presentation and immune cell function were identified. Furthermore, this analysis showed that genes influenced by IRF1 and IRF2 could be grouped into multiple classes. Under basal conditions, there was a subset of antigen presentation-related genes whose expression was activated by IRF2 (e.g., TAP2, ERAP1), others that were repressed by IRF2 and remained so in the double knockouts (e.g., H2-T9), and yet others that were repressed by IRF2 but did not remain so in the double knockouts (e.g., PSMB8, PSMB10) (FIG. 5D). After stimulating with IFNγ, some genes were primarily activated by IRF1 (e.g., PSME1, PSME2, PSMB9), others were primarily repressed by IRF2 (e.g., PD-L1), and yet others were activated by both IRF1 and IRF2 (e.g., TAP2, ERAP1) (FIG. 5E). Collectively, these studies demonstrate that while some genes are acted on antagonistically by IRF1 and IRF2, other genes are regulated synergistically by these two transcription factors and that the relative contributions of IRF1 vs. IRF2 in mediating these expression changes varies depending on the inflammatory state of the cell.

### Example 5

### IRF2 in Cancer

Given that experimentally-induced loss of IRF2 both compromises MHC-I presentation and increases PD-L1 expression, it was of interest to see how often IRF2 is downregulated in primary cancers. We used the TIMER bioinformatics tool²⁹ to mine publicly available databases for IRF2 expression in primary human cancers. Remarkably,

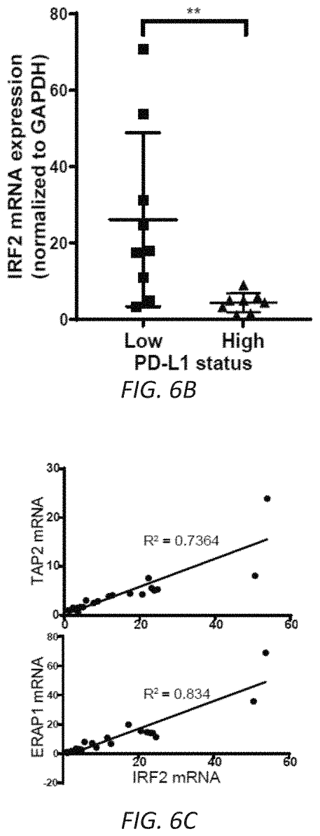

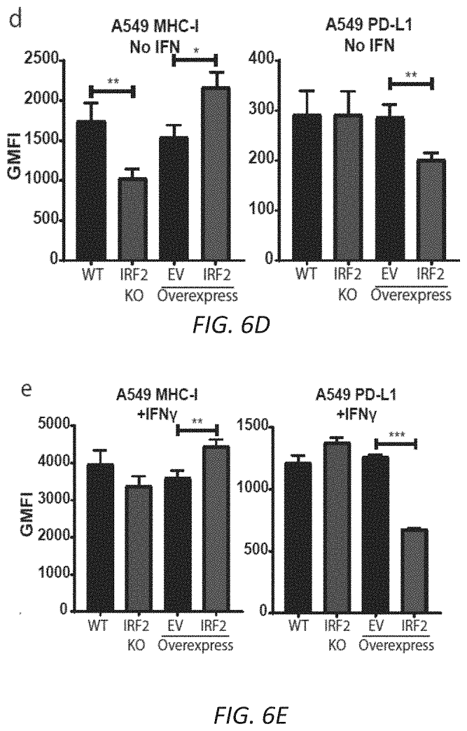

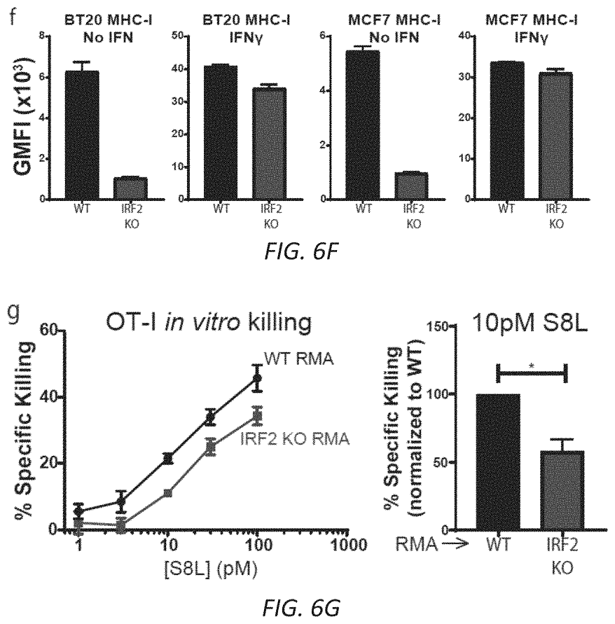

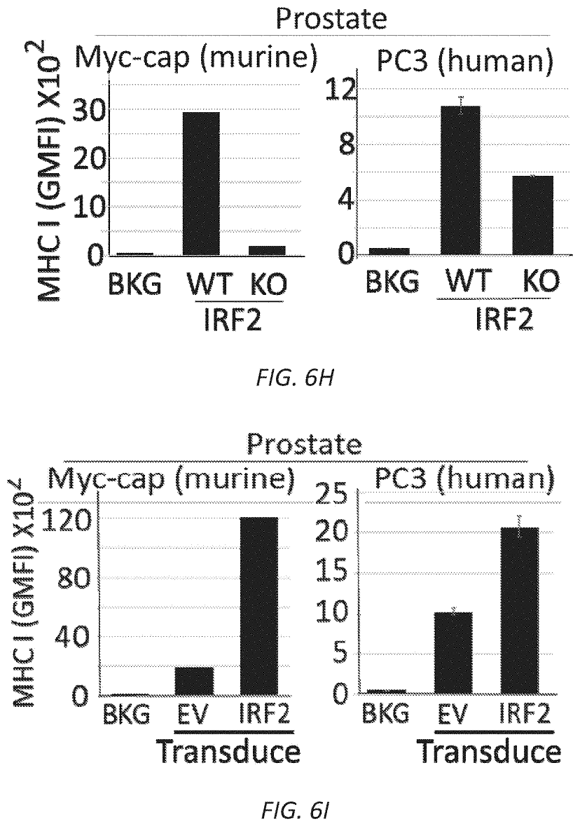

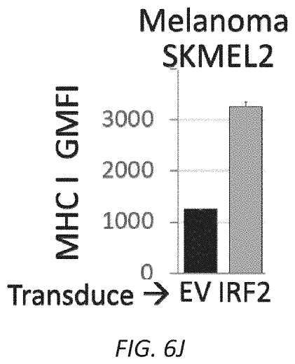

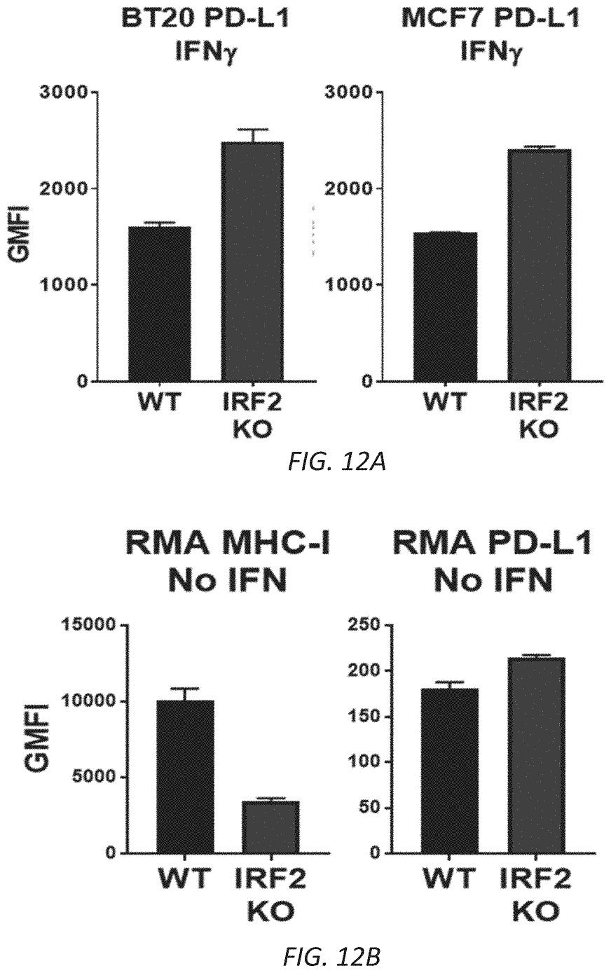

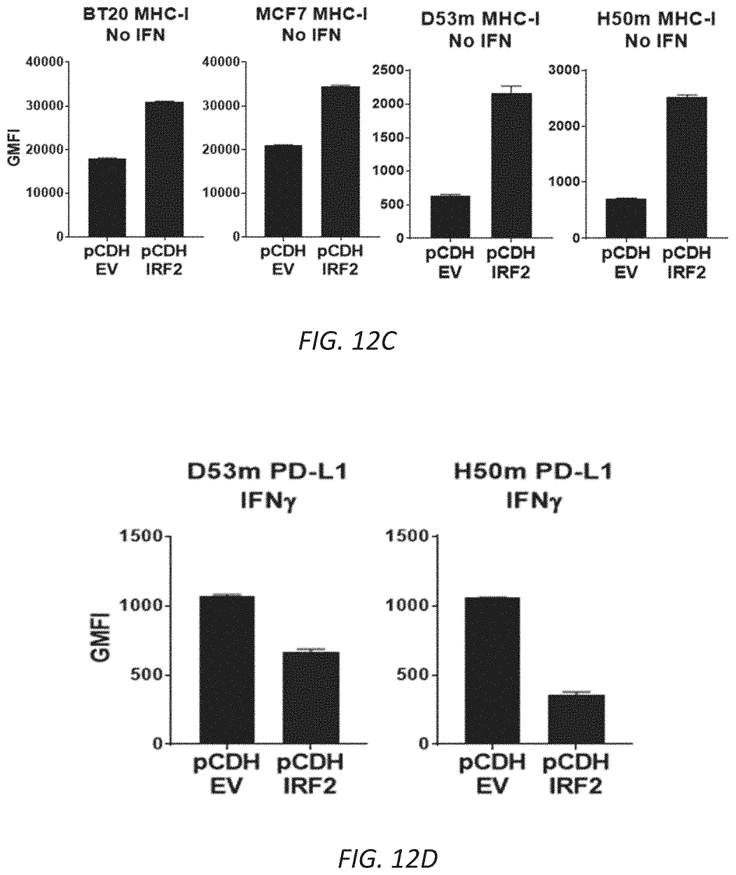

IRF2 was downregulated in several kinds of human cancers and the overall reductions were highly statistically significant (FIG. 6A). For each of the IRF2-low cancers, which included invasive breast carcinoma, cholangiocarcinoma, colon adenocarcinoma, liver hepatocellular carcinoma, lung adenocarcinoma and squamous cell carcinoma (non-small cell lung cancers), prostate, rectum and stomach adenocarcinomas, and uterine corpus endometrial carcinoma, a subset of patients had very low levels of IRF2. We chose one of the IRF2-low cancers, non-small cell lung cancer (NSCLC), to determine whether IRF2 levels were functionally limiting in primary cancers. At our institution, NSCLCs were screened for PD-L1 expression by immunohistochemistry (IHC) at the time of diagnosis, which enabled us to randomly select tumors spanning a spectrum of PD-L1 expression. We extracted RNA from archival patient biopsy material and quantified expression of IRF2, TAP2, and ERAP1 by qPCR. In these lung cancers, IRF2 mRNA levels and PD-L1 IHC status were significantly inversely correlated (FIG. 6B). Additionally, consistent with our cell line findings, TAP2 and ERAP1 mRNA levels positively and significantly correlated with IRF2 mRNA levels (FIG. 6C). To formally test cause and effect for these correlations, we analyzed a human NSCLC cell line, A549, that is IRF2-low relative to other NSCLCs tested in the NCI-60 pane1³⁰. A549 cells are also MHC-I-low and PD-L1-positive^{31,32}. Eliminating the residual IRF2 in A549 cells by CRISPR-Cas9-mediated knockout further decreased surface MHC-I but did not further increase surface PD-L1 (FIG. 6D). In contrast, restoring IRF2 by transfection repressed surface PD-L1 expression and increased surface MHC-I expression (FIG. 6D). Stimulation of A549 with IFNγ augmented both surface MHC-I and PD-L1 expression, as expected, but transfection of IRF2 still had the same pattern of effects as without IFN (repressing PD-L1 and further increasing MHC-I expression) (FIG. 6E). To further generalize these findings to other cancers, we analyzed two human breast cancers, human prostate cancer, and human melanoma, as well as two mouse sarcomas, a mouse lymphoma, and a mouse prostate cancer, and found similar results (FIGS. 6F-J, FIGS. 12A-D).

The effects of IRF2 loss on antigen presentation and checkpoint inhibition are predicted to make it harder for CD8⁺ T cells to kill IRF2-low cells. To test this and quantify the magnitude of the effect, we analyzed mouse wild-type vs. IRF2-KO lymphoma (RMA) cell lines. RMA cells were chosen because they are known to be very good targets for cytotoxic T cell killing assays (and are, therefore, a stringent test) and express H2-K^{b}, which allows the use of potent CD8⁺ T cell effectors from the H2-K^{b}-S8L-specific TCR transgenic OT-I model. Pre-activated OT-I effectors were cultured with pairs of wild-type or IRF2-KO cells that were S8L-pulsed or not and labeled with different amounts of the dye CF SE. After 4 hours, specific killing was quantified by flow cytometry and the dose-response curve for the IRF2-KO cells was shifted up about 3-fold (which equates to a decreased killing efficiency of about 67%) in the IRF2-KO RMA, as compared to the wild-type RMA (FIG. 6G), demonstrating that tumor cells lacking IRF2 are harder for CD8⁺ T cells to eliminate. Taken together, these findings show that IRF2 downregulation leads to immune evasion and that there are several types of human cancers which may use this escape mechanism.

### Example 6

### Epigenetic Modifying Drugs Can Reverse IRF2 Down Regulation in Tumor Cells

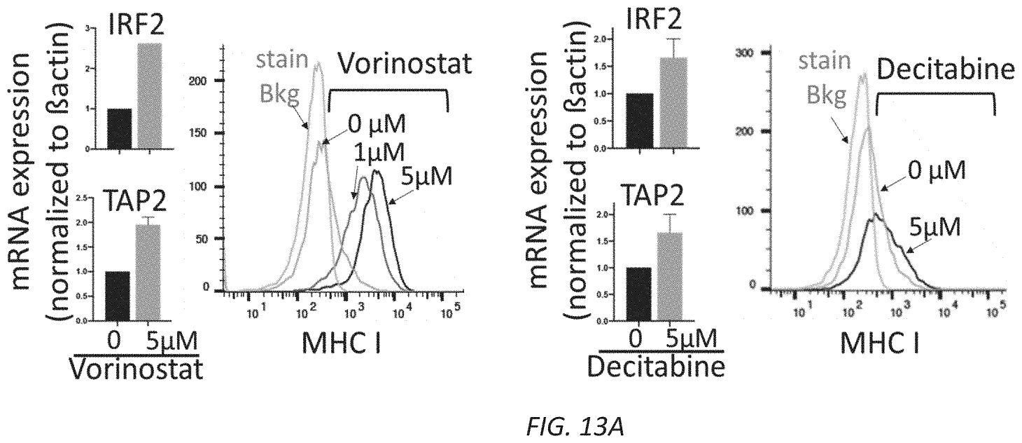

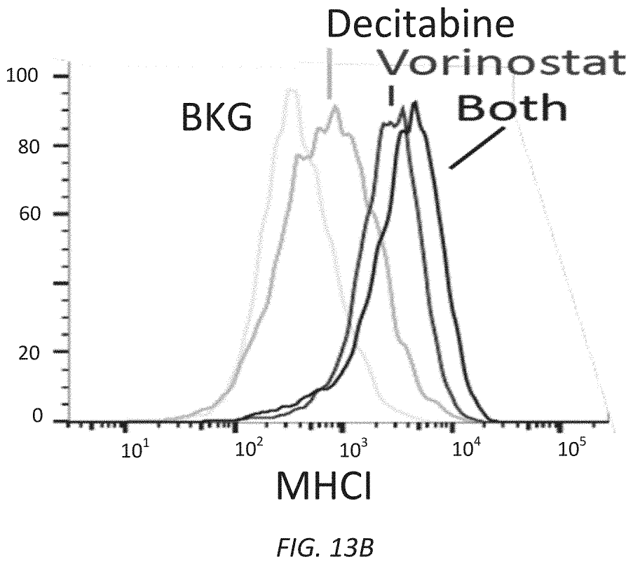

We tested two epigenetic modifiers on an IRF2low, MHC I low murine cancer cell line (F221 MCA-sarcoma). F221 murine sarcoma cells were treated with Vorinostat (1 uM or 5 uM), Decitabine (5 uM), or control diluent (DMSO) with no drug for 24 hrs and then assayed for IRF2 and TAP2 mRNA levels by qPCR and MHC I protein levels by flow cytometry. Remarkably, both Vorinostat (an HDAC inhibitor) and Decitabine (a hypomethylating agent) increased expression of IRF2 mRNA, mRNA for IRF2's target gene, TAP2, and surface MHC I (H-2Kb) protein levels (FIG. 13B). These data provide evidence that cancers downregulate IRF2 expression by epigenetic silencing. Moreover, the data show that epigenetic modifying drugs can reverse IRF2 down regulation and result in increased expression of IRF2-dependent genes (TAP2) and surface expression of MHC I molecules.

In addition, F221 MCA-sarcoma cells were treated with a combination of Vorinostat (5 uM) and/or Decitabine (5 uM) for 24 hrs and then surface expression of MEW I molecules were quantified by flow cytometry. As shown FIG. 13B, in the combination of the two drugs was tested and found to have at least an additive effect.

### REFERENCES

1. Kaplan, D. H., et al. Demonstration of an interferon gamma-dependent tumor surveillance system in immunocompetent mice. Proc Natl Acad Sci U S A 95, 7556-7561 (1998).

2. Street, S. E., Cretney, E. & Smyth, M. J. Perforin and interferon-gamma activities independently control tumor initiation, growth, and metastasis. Blood 97, 192-197 (2001).

3. Shankaran, V., et al. IFNgamma and lymphocytes prevent primary tumour development and shape tumour immunogenicity. Nature 410, 1107-1111 (2001).

4. Svane, I. M., et al. Chemically induced sarcomas from nude mice are more immunogenic than similar sarcomas from congenic normal mice. Eur J Immunol 26, 1844-1850 (1996).

5. Engel, A. M., Svane, I. M., Rygaard, J. & Werdelin, O. MCA sarcomas induced in scid mice are more immunogenic than MCA sarcomas induced in congenic, immunocompetent mice. Scand J Immunol 45, 463-470 (1997).

6. Mittal, D., Gubin, M. M., Schreiber, R. D. & Smyth, M. J. New insights into cancer immunoediting and its three component phases—elimination, equilibrium and escape. Curr Opin Immunol 27, 16-25 (2014).

7. Sabbatino, F., et al. PD-L1 and HLA Class I Antigen Expression and Clinical Course of the Disease in Intrahepatic Cholangiocarcinoma. Clin Cancer Res 22, 470-478 (2016).

8. Gettinger, S., et al. Impaired HLA Class I Antigen Processing and Presentation as a Mechanism of Acquired Resistance to Immune Checkpoint Inhibitors in Lung Cancer. Cancer Discov 7, 1420-1435 (2017).

9. Abele, R. & Tampe, R. The ABCs of immunology: structure and function of TAP, the transporter associated with antigen processing. Physiology (Bethesda) 19, 216-224 (2004).

10. Saric, T., et al. An IFN-gamma-induced aminopeptidase in the ER, ERAP1, trims precursors to MHC class I-presented peptides. Nat Immunol 3, 1169-1176 (2002).

11. Serwold, T., Gonzalez, F., Kim, J., Jacob, R. & Shastri, N. ERAAP customizes peptides for MEW class I molecules in the endoplasmic reticulum. Nature 419, 480-483 (2002).

12. Zamora, A. E., Crawford, J. C. & Thomas, P. G. Hitting the Target: How T Cells Detect and Eliminate Tumors. J Immunol 200, 392-399 (2018).

13. Shen, Z., Reznikoff, G., Dranoff, G. & Rock, K. L. Cloned dendritic cells can present exogenous antigens on both MHC class I and class II molecules. J Immunol 158, 2723-2730 (1997).

14. Brinkman, E. K., Chen, T., Amendola, M. & van Steensel, B. Easy quantitative assessment of genome editing by sequence trace decomposition. Nucleic Acids Res 42, e168 (2014).

15. Masumi, A., Yamakawa, Y., Fukazawa, H., Ozato, K. & Komuro, K. Interferon regulatory factor-2 regulates cell growth through its acetylation. J Biol Chem 278, 25401-25407 (2003).

16. Porgador, A., Yewdell, J. W., Deng, Y., Bennink, J. R. & Germain, R. N. Localization, quantitation, and in situ detection of specific peptide-MHC class I complexes using a monoclonal antibody. Immunity 6, 715-726 (1997).

17. Fernandes, D. M., Vidard, L. & Rock, K. L. Characterization of MEW class II-presented peptides generated from an antigen targeted to different endocytic compartments. Eur J Immunol 30, 2333-2343 (2000).

18. Dong, H., et al. Tumor-associated B7-H1 promotes T-cell apoptosis: a potential mechanism of immune evasion. Nat Med 8, 793-800 (2002).

19. Yu, H., Boyle, T. A., Zhou, C., Rimm, D. L. & Hirsch, F. R. PD-L1 Expression in Lung Cancer. J Thorac Oncol 11, 964-975 (2016).

20. Tanaka, N., Kawakami, T. & Taniguchi, T. Recognition DNA sequences of interferon regulatory factor 1 (IRF-1) and IRF-2, regulators of cell growth and the interferon system. Mol Cell Biol 13, 4531-4538 (1993).

21. Darnell, J. E., Jr., Kerr, I. M. & Stark, G. R. Jak-STAT pathways and transcriptional activation in response to IFNs and other extracellular signaling proteins. Science 264, 1415-1421 (1994).

22. Harada, H., et al. Structurally similar but functionally distinct factors, IRF-1 and IRF-2, bind to the same regulatory elements of IFN and IFN-inducible genes. Cell 58, 729-739 (1989).

23. Watanabe, N., Sakakibara, J., Hovanessian, A. G., Taniguchi, T. & Fujita, T. Activation of IFN-beta element by IRF-1 requires a posttranslational event in addition to IRF-1 synthesis. Nucleic Acids Res 19, 4421-4428 (1991).

24. Ren, G., Cui, K., Zhang, Z. & Zhao, K. Division of labor between IRF1 and IRF2 in regulating different stages of transcriptional activation in cellular antiviral activities. Cell Biosci 5, 17 (2015).

25. Oshima, S., et al. Interferon regulatory factor 1 (IRF-1) and IRF-2 distinctively up-regulate gene expression and production of interleukin-7 in human intestinal epithelial cells. Mol Cell Biol 24, 6298-6310 (2004).

26. Hobart, M., Ramassar, V., Goes, N., Urmson, J. & Halloran, P. F. IFN regulatory factor-1 plays a central role in the regulation of the expression of class I and II MHC genes in vivo. J Immunol 158, 4260-4269 (1997).

27. Lee, S. J., et al. Interferon regulatory factor-1 is prerequisite to the constitutive expression and IFN-gamma-induced upregulation of B7-H1 (CD274). FEBS Lett 580, 755-762 (2006).

28. Garcia-Diaz, A., et al. Interferon Receptor Signaling Pathways Regulating PD-L1 and PD-L2 Expression. Cell Rep 19, 1189-1201 (2017).

29. Li, B., et al. Comprehensive analyses of tumor immunity: implications for cancer immunotherapy. Genome Biol 17, 174 (2016).

30. Rajapakse, V. N., et al. CellMinerCDB for Integrative Cross-Database Genomics and Pharmacogenomics Analyses of Cancer Cell Lines. iScience 10, 247-264 (2018).

31. Teo, J., et al. A preliminary study for the assessment of PD-L1 and PD-L2 on circulating tumor cells by microfluidic-based chipcytometry. Future Sci OA 3, FS0244 (2017).

32. Redondo, M., et al. Altered HLA class I expression in non-small cell lung cancer is independent of c-myc activation. Cancer Res 51, 2463-2468 (1991).

33. Carretero, R., et al. Analysis of HLA class I expression in progressing and regressing metastatic melanoma lesions after immunotherapy. Immunogenetics 60, 439-447 (2008).

34. del Campo, A. B., et al. Immune escape of cancer cells with beta2-microglobulin loss over the course of metastatic melanoma. Int J Cancer 134, 102-113 (2014).

35. Marincola, F. M., Jaffee, E. M., Hicklin, D. J. & Ferrone, S. Escape of human solid tumors from T-cell recognition: molecular mechanisms and functional significance. Adv Immunol 74, 181-273 (2000).

36. Hicklin, D. J., Marincola, F. M. & Ferrone, S. HLA class I antigen downregulation in human cancers: T-cell immunotherapy revives an old story. Mol Med Today 5, 178-186 (1999).

37. Zou, W. & Chen, L. Inhibitory B7-family molecules in the tumour microenvironment. Nat Rev Immunol 8, 467-477 (2008).

38. Pardoll, D. M. The blockade of immune checkpoints in cancer immunotherapy. Nat Rev Cancer 12, 252-264 (2012).

39. Wang, X., et al. Effectiveness and safety of PD-1/PD-L1 inhibitors in the treatment of solid tumors: a systematic review and meta-analysis. Oncotarget 8, 59901-59914 (2017).

40. Dorand, R.D., et al. Cdk5 disruption attenuates tumor PD-L1 expression and promotes antitumor immunity. Science 353, 399-403 (2016).

41. Wu, A., et al. Loss of VGLL4 suppresses tumor PD-L1 expression and immune evasion. EMBO J 38(2019).

42. Jongsma, M. L. M., Guarda, G. & Spaapen, R. M. The regulatory network behind MHC class I expression. Mol Immunol (2017).

43. Kincaid, E. Z., et al. Mice completely lacking immunoproteasomes show major changes in antigen presentation. Nat Immunol 13, 129-135 (2011).

44. Jesse, T. L., LaChance, R., Iademarco, M. F. & Dean, D. C. Interferon regulatory factor-2 is a transcriptional activator in muscle where It regulates expression of vascular cell adhesion molecule-1. J Cell Biol 140, 1265-1276 (1998).

45. Vaughan, P. S., et al. Activation of a cell-cycle-regulated histone gene by the oncogenic transcription factor IRF-2. Nature 377, 362-365 (1995).

46. Rock, K. L., Rothstein, L. & Gamble, S. Generation of class I WIC-restricted T-T hybridomas. J Immunol 145, 804-811 (1990).

47. Sanjana, N. E., Shalem, O. & Zhang, F. Improved vectors and genome-wide libraries for CRISPR screening. Nat Methods 11, 783-784 (2014).

48. Shalem, O., et al. Genome-scale CRISPR-Cas9 knockout screening in human cells. Science 343, 84-87 (2014).

49. Hearn, A., York, I. A. & Rock, K. L. The specificity of trimming of MEW class I-presented peptides in the endoplasmic reticulum. J Immunol 183, 5526-5536 (2009).

50. Bolger, A. M., Lohse, M. & Usadel, B. Trimmomatic: a flexible trimmer for Illumina sequence data. Bioinformatics 30, 2114-2120 (2014).

51. Li, B. & Dewey, C. N. RSEM: accurate transcript quantification from RNA-Seq data with or without a reference genome. BMC Bioinformatics 12, 323 (2011).

52. Pruitt, K. D., Tatusova, T., Brown, G. R. & Maglott, D. R. NCBI Reference Sequences (RefSeq): current status, new features and genome annotation policy. Nucleic Acids Res 40, D130-135 (2012).

53. Johnson, W. E., Li, C. & Rabinovic, A. Adjusting batch effects in microarray expression data using empirical Bayes methods. Biostatistics 8, 118-127 (2007).

54. Leek, J. T., Johnson, W. E., Parker, H. S., Jaffe, A. E. & Storey, J. D. The sva package for removing batch effects and other unwanted variation in high-throughput experiments. Bioinformatics 28, 882-883 (2012).

55. Love, M. I., Huber, W. & Anders, S. Moderated estimation of fold change and dispersion for RNA-seq data with DESeq2. Genome Biol 15, 550 (2014).

56. Mould, A. W., Morgan, M. A., Nelson, A. C., Bikoff, E. K. & Robertson, E. J. Blimp1/Prdm1 Functions in Opposition to Irf1 to Maintain Neonatal Tolerance during Postnatal Intestinal Maturation. PLoS Genet 11, e1005375 (2015).

57. Doody, G. M., Stephenson, S., McManamy, C. & Tooze, R. M. PRDM1/BLIMP-1 modulates IFN-gamma-dependent control of the MEW class I antigen-processing and peptide-loading pathway. J Immunol 179, 7614-7623 (2007).

58. Lu, C., Redd, P. S., Lee, J. R., Savage, N. & Liu, K. The expression profiles and regulation of PD-L1 in tumor-induced myeloid-derived suppressor cells. Oncoimmunology 5, e1247135 (2016).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject who has cancer, the method comprising:
providing a sample comprising cells from the cancer;
detecting a level of a biomarker selected from the group consisting of interferon regulatory factor 2 (IRF2); transporter 2, ATP binding cassette subfamily B membertransporter 2, ATP binding cassette subfamily B member (TAP2); and endoplasmic reticulum aminopeptidase 1 (ERAP1) in the sample;
comparing the level of the biomarker to a reference level; and
(i) identifying a subject as having biomarker levels above the reference level, and treating the subject with a checkpoint inhibitor;
(ii) identifying a subject as having biomarker levels below the reference level, and treating the subject with a checkpoint inhibitor and an interferon inducer or epigenetic modifier; or
(iii) identifying a subject as having biomarker levels below the reference level, and treating the subject with a treatment that does not include a checkpoint inhibitor.

2. (canceled)

3. (canceled)

4. (canceled)

5. A method of treating a subject with cancer with a checkpoint inhibitor, the method comprising providing a sample comprising cells from the cancer;
detecting a level of a biomarker selected from the group consisting of interferon regulatory factor 2 (IRF2); transporter 2, ATP binding cassette subfamily B membertransporter 2, ATP binding cassette subfamily B member (TAP2); and endoplasmic reticulum aminopeptidase 1 (ERAP1) in the sample;
comparing the level of the biomarker to a reference level; and
treating the subject with the checkpoint inhibitor if the level of the biomarker in the sample is above the reference level.

6. The method of claim 1, further comprising:
detecting a level of programmed cell death 1 ligand 1 (PD-L1) in the sample;
comparing the level of PD-L1 to a reference level; and
treating the subject with the checkpoint inhibitor if the level of the biomarker in the sample is above the reference level, and the level of PD-L1 is above the reference level, or treating the subject with the checkpoint inhibitor and an interferon inducer or epigenetic modifier if the level of the biomarker in the sample is below the reference level, and the level of PD-L1 is above the reference level, or
treating the subject with a treatment that does not include a checkpoint inhibitor if the level of the biomarker in the sample is below the reference level, and the level of PD-L1 is below the reference level.

7. The method of claim 1, wherein detecting a level of the biomarker in the sample comprises measuring mRNA or protein levels in the sample.

8. The method of claim 7, wherein measuring mRNA comprises using quantitative PCR.

9. The method of claim 1, wherein the subject has a carcinoma or adenocarcinoma.

10. The method of claim 9, wherein the carcinoma or adenocarcinoma is breast carcinoma, cholangiocarcinoma, colon adenocarcinoma, liver hepatocellular carcinoma, lung adenocarcinoma, squamous cell carcinoma (non-small cell lung cancer), prostate adenocarcinoma, rectum adenocarcinoma, stomach adenocarcinomas, or uterine carcinoma.

11. The method of claim 1, wherein the checkpoint inhibitor is an antibody that targets programmed cell death protein 1 (PD-1), PD-Ligand 1 (PD-L1), PDL2, or cytotoxic T-lymphocyte-associated protein 4 (CTLA-4).

12. The method of claim 1, wherein the interferon inducer is type I or type 2 interferon.

13. The method of claim 12, wherein the interferon is interferon alpha-2b, PEGylated interferon alpha-2b, PEGylated interferon-alpha-2a, Human leukocyte Interferon-alpha (HuIFN-alpha-Le), Interferon beta 1a, Interferon beta 1b, or Interferon gamma (e.g., IFN-gamma 1b).

14. The method of claim 1, wherein the interferon inducer is poly(I:C), Poly(A:U), ampligen [poly(I)-poly(Cl2U)], polyICLC), or Imiquimod.

15. The method of claim 1, wherein the epigenetic modifier is a DNA methyltransferase (DNMT) inhibitor or a Histone deacetylase (HDAC) inhibitor.

16. The method of claim 15, comprising administering a DNMT inhibitor and an HDAC inhibitor.

17. The method of claim 15, wherein the HDAC inhibitor is Suberoylanilide hydroxamic acid (SAHA/Vorinostat/Zolinza), Trichostatin A (TSA), belinostat (PXD101), depsipeptide (FK228/romidepsin/ISTODAX), Entinostat (SNDX-275), mocetinostat (MGCD0103), valproic acid, Sodium phenylbutyrate, LAQ824, panobinostat (LBH589), entinostat (MS275), CI-994 (N-acetyldinaline/tacedinaline), EVP-0334, SRT501, CUDC-101, JNJ-26481585, PCI24781, or Givinostat (ITF2357).

18. The method of claim 15, wherein the DNMT inhibitor is 5′-azacytidine (Aza), Decitabine, Cladribine, Fludarabine, Clofarabine, Procainamide, Procaine, Zebularine (1-(β-D-ribofuranosyl)-1,2-dihydropyrimidin-2-one), (−)-epigallocatechin-3-gallate, MG98, hydralazine, RG108, or chlorogenic acid.


SEQUENCE LISTING (36 sequences)

SEQ ID NO: 1 - DNA, 20 bp, Artificial Sequence
  sgRNA sequence Mouse B2m
agtatactcacgccacccac

SEQ ID NO: 2 - DNA, 20 bp, Artificial Sequence
  sgRNA sequence mouse TAP1
actaatggactcgcacacgt

SEQ ID NO: 3 - DNA, 20 bp, Artificial Sequence
  sgRNA sequence mouse TAP2
attacacgacccgaatagcg

SEQ ID NO: 4 - DNA, 20 bp, Artificial Sequence
  sgRNA sequence mouse ERAP1
tgcagcatccagagcataat

SEQ ID NO: 5 - DNA, 20 bp, Artificial Sequence
  sgRNA sequence mouse IRF2
tccgaacgaccttccaagaa

SEQ ID NO: 6 - DNA, 20 bp, Artificial Sequence
  sgRNA sequence mouse IRF1
ctcatccgcattcgagtgat

SEQ ID NO: 7 - DNA, 20 bp, Artificial Sequence
  sgRNA sequence human IRF2
tgcatgcggctagacatggg

SEQ ID NO: 8 - DNA, 25 bp, Artificial Sequence
  Primer Mouse B2m F
caccgagtatactcacgccacccac

SEQ ID NO: 9 - DNA, 25 bp, Artificial Sequence
  Primer Mouse B2m R
aaacgtgggtggcgtgagtatactc

SEQ ID NO: 10 - DNA, 25 bp, Artificial Sequence
  Primer Mouse TAP1 F
caccgactaatggactcgcacacgt

SEQ ID NO: 11 - DNA, 25 bp, Artificial Sequence
  Primer Mouse TAP1 R
aaacacgtgtgcgagtccattagtc

SEQ ID NO: 12 - DNA, 25 bp, Artificial Sequence
  Primer Mouse TAP2 F
caccgattacacgacccgaatagcg

SEQ ID NO: 13 - DNA, 25 bp, Artificial Sequence
  Primer Mouse TAP2 R
aaaccgctattcgggtcgtgtaatc

SEQ ID NO: 14 - DNA, 25 bp, Artificial Sequence
  Primer Mouse ERAP1 F
caccgtgcagcatccagagcataat

SEQ ID NO: 15 - DNA, 25 bp, Artificial Sequence
  Primer Mouse ERAP1 R
aaacattatgctctggatgctgcac

SEQ ID NO: 16 - DNA, 25 bp, Artificial Sequence
  Primer Mouse IRF2 F
caccgtccgaacgaccttccaagaa

SEQ ID NO: 17 - DNA, 25 bp, Artificial Sequence
  Primer Mouse IRF2 R
aaacttcttggaaggtcgttcggac

SEQ ID NO: 18 - DNA, 25 bp, Artificial Sequence
  Primer Mouse IRF1 F
caccgctcatccgcattcgagtgat

SEQ ID NO: 19 - DNA, 25 bp, Artificial Sequence
  Primer Mouse IRF1 R
aaacatcactcgaatgcggatgagc

SEQ ID NO: 20 - DNA, 25 bp, Artificial Sequence
  Primer Human IRF2 F
caccgtgcatgcggctagacatggg

SEQ ID NO: 21 - DNA, 25 bp, Artificial Sequence
  Primer Human IRF2 R
aaaccccatgtctagccgcatgcac

SEQ ID NO: 22 - DNA, 21 bp, Artificial Sequence
  primer hU6-F
gagggcctatttcccatgatt

SEQ ID NO: 23 - DNA, 34 bp, Artificial Sequence
  Human IRF2 AgeI fwd
gactaccggtatgccggtggaaaggatgcgcatg

SEQ ID NO: 24 - DNA, 52 bp, Artificial Sequence
  Human IRF2 sgRNA mut rev
gccgtgcctcgctgcgtgcatccaggggatctgaaaaatcttcttttccttg

SEQ ID NO: 25 - DNA, 57 bp, Artificial Sequence
  Human IRF2 sgRNA mut fwd
gatgcacgcagcgaggcacggctgggatgtggaaaaagatgcaccactctttagaaa

SEQ ID NO: 26 - DNA, 35 bp, Artificial Sequence
  Human IRF2 MluI rev
gatcacgcgtttaacagctcttgacgcgggcctgg

SEQ ID NO: 27 - DNA, 34 bp, Artificial Sequence
  Mouse IRF2 AgeI fwd
gatcaccggtatgccggtggaacggatgcgaatg

SEQ ID NO: 28 - DNA, 54 bp, Artificial Sequence
  Mouse IRF2 sgRNA mut rev
ccttttttcgaggggcgctctgataagggcagcatccggtagactctgaaggcg

SEQ ID NO: 29 - DNA, 57 bp, Artificial Sequence
  Mouse IRF2 sgRNA mut fwd
cttatcagagcgcccctcgaaaaaaggaaagaaaccaaagacagaaaaagaagagag

SEQ ID NO: 30 - DNA, 35 bp, Artificial Sequence
  Mouse IRF2 MluI rev
gatcacgcgtttaacagctcttgacacgggcctgg

SEQ ID NO: 31 - DNA, 20 bp, Artificial Sequence
  Primer Mouse TAP2 F
caaattgacaggcgccatct

SEQ ID NO: 32 - DNA, 23 bp, Artificial Sequence
  Primer Mouse TAP2 R
gcttcttctcaaactggatctcc

SEQ ID NO: 33 - DNA, 23 bp, Artificial Sequence
  Primer Mouse ERAP1 F
cttaggcttgctctcttttagcg

SEQ ID NO: 34 - DNA, 20 bp, Artificial Sequence
  Primer Mouse ERAP1 R
gactcctgctcccgatcctc

SEQ ID NO: 35 - DNA, 24 bp, Artificial Sequence
  Primer Mouse PD-L1 F
caagaaagctaatgcaggtttcac

SEQ ID NO: 36 - DNA, 21 bp, Artificial Sequence
  Primer Mouse PD-L1 R
cctgcggatgactttagagtc